(19) United States
(12) Patent Application Publication
(10) Pub. No.: US 2025/0131214 A1
(43) Pub. Date: Apr. 24, 2025

(54) METHODS, DEVICES AND SYSTEMS FOR ANALYZING AND CONVERTING SPECIALIZED LANGUAGE

(71) Applicant: PLAINSPEAK HEALTH, LLC, Alexandria, VA (US)

(72) Inventor: David C. BOWEN, Alexandria, VA (US)

(73) Assignee: PLAINSPEAK HEALTH, LLC, Alexandria, VA (US)

(21) Appl. No.: 18/919,908

(22) Filed: Oct. 18, 2024

Related U.S. Application Data
(60) Provisional application No. 63/591,571, filed on Oct. 19, 2023.

(51) Int. Cl. G06F 40/58; G16H 10/60; G16H 80/00

(52) U.S. Cl. CPC G06F 40/58; G16H 10/60; G16H 80/00

(57) ABSTRACT

Patients often leave medical appointments with misunderstandings about their diagnoses and treatment plans due to the specialized language used by healthcare professionals. This miscommunication can lead to serious health complications, longer wait times for care, and increased medical costs. This disclosure provides an affordable, scalable solution to this problem through a system that uses natural language processing and large language models to translate medical jargon into plain language that patients can easily understand. The system also prompts patients to ask informed questions and connects them with others who share similar health experiences. Additionally, it simplifies insurance documents, helping patients better understand their coverage and treatment options. By addressing these communication barriers, the system aims to improve health outcomes, enhance treatment adherence, and reduce overall healthcare costs for patients and health systems.

### CROSS REFERENCE TO RELATED APPLICATIONS

This U.S. patent application claims the benefit of U.S. Provisional Application No. 63/591,571, filed Oct. 19, 2023, entitled METHODS, DEVICES AND SYSTEMS FOR ANALYZING SPECIALIZED LANGUAGE AND CONVERTING THE ANALYZED SPECIALIZED LANGUAGE INTO A BROADLY UNDERSTOOD LANGUAGE, AND ENABLING A SOCIAL NETWORK BASED ON SHARED CONDITIONS OR INTERESTS which is hereby incorporated by reference in its entirety.

### TECHNICAL FIELD

The disclosure relates to a communication system, and more specifically, to a communication system for analyzing and translating specialized language (e.g., specialized terminology) into broadly understood language (e.g., commonly understood language). The system further enables the formation of a social network based on shared health conditions or interests.

### BACKGROUND

The Irish writer, George Bernard Shaw, once said: “The single biggest problem in communication is the illusion that it has taken place.” That sentiment is often true in health care, where medical professionals are trained to convey information in dense, jargon-filled specialized language, whereas patients generally only use broadly used or plain language.

Doctors may believe that a conversation with their patient that uses specialized language results in clear communication with the patient (e.g., a common understanding or meeting of the minds between the medical professional and the patient). However, the reality is that the doctor likely has not communicated in a manner that the patient has understood. This can result in a situation where the doctor believes s/he has communicated clearly about the medications or actions a patient is supposed to take as a result of their medical condition, but where the patient does not clearly understand the information that the doctor is trying to convey, due to the complexity of the language used, and thus either fails to take the action the doctor recommends or takes a different and mistaken action, based on the miscommunication between doctor and patient.

A study conducted by New York Presbyterian Hospital found that 75% of orthopedic surgeons believed they had communicated successfully with their patients—but only 21% of patients felt they had received clear communication. Tongue et al. (2005). Communication skills for patient-centered care: Research-based, easily learned techniques for medical interviews that benefit orthopedic surgeons and their patients. The Journal of Bone and Joint Surgery. 87A. 652-658. 10.2106/00004623-200503000-00027. In a similar study conducted by Yale University, nearly every physician (98%) reported having at least occasionally discussed the fears and anxieties of their patients, but over half of patients reported never having had any such discussion. Olson et al. (2010). Communication Discrepancies Between Physicians and Hospitalized Patients. Archives of Internal Medicine. 170. 1302-7. 10.1001/archinternmed.2010.239.

There is often an illusion in health care that communication has taken place, but the reality is that patients misunderstand the information their doctors provide. According to the National Action Plan to Improve Health Literacy, “Nearly 9 out of 10 adults have difficulty using the everyday health information that is routinely available in our health care facilities, retail outlets, media and communities.” National Action Plan to Improve Health Literacy, U.S. Department of Health and Human Services, Office of Disease Prevention and Health Promotion, p. 1 (2010). Even for people with strong general literacy, the stressful and jargon-filled environment of a medical visit can be daunting and can impair understanding. If patients are unable to understand their diagnosis or prescribed treatments, the patients likely may not participate meaningfully in their own care, leading to poorer health outcomes. More than a third of all Americans have only basic or below-basic health literacy, meaning that they will struggle to understand information presented in complex medical terms. Individuals from communities of color, seniors, and persons who speak English as a second language are particularly likely to have low health literacy, adding to health disparities.

In one recent study to assess how well patients understood (or failed to understand) medical jargon, researchers asked patients to describe what was meant by several different medical terms, describing conditions such as infections or instructions such as a prohibition of eating and drinking before surgery. In general, comprehension was poor. For some terms, fewer than 1 in 20 patients correctly understood the term. Gotlieb et al. (2022). Accuracy in Patient Understanding of Common Medical Phrases. JAMA Network Open. 5. e2242972. 10.1001/jamanetworkopen.2022.42972.

The result of such widespread failure to communicate is that crucial information is missed, and patients either take incorrect actions or fail to take correct actions to improve their health. According to a study from the Columbia University School of Nursing, “Patients who are unable to successfully interpret health information have increased hospitalization rates, develop more diseases, and experience higher mortality.” Hickey et al. (2018). Low health literacy: Implications for managing cardiac patients in practice. The Nurse practitioner. 43. 49-55. 10.1097/01.NPR.0000541468.54290.49.

In a study of patients with heart failure, low health literacy was associated with increased risk of visits to the Emergency Department, hospitalization, and death. Nor are these findings specific to the USA. In a review of studies conducted in multiple countries, lower parental literacy was associated with poorer health outcomes for children in rich and poor countries alike. Zaidman et al. (2022). Impact of parental health literacy on the health outcomes of children with chronic disease globally: A systematic review. Journal of Paediatrics and Child Health. 59. 10.1111/jpc.16297.

Solutions proposed to the health problems created by medical miscommunication have ranged from the individual to the societal. Some studies have found that high-touch and expensive interventions, such as using community health workers to provide individualized comprehension services to patients, can improve comprehension of medical information. Butayeva et al. (2023). The impact of health literacy interventions on glycemic control and self-management outcomes among type 2 diabetes mellitus: A systematic review. Journal of Diabetes. 2023; 15(9): 724-735. doi:10.1111/1753-0407.13436.

However, these kinds of labor-intensive interventions are unlikely to be broadly scalable in health systems that have chronic staff shortages and are trying to contain costs.

Focusing on the other half of the communication dyad, there are also proposals to improve the communication skills of doctors, either through re-training courses for established professionals, or by incorporating communication skills into medical school training. Even if successful, these initiatives are likely to take many years to achieve real change.

The same is true for proposals to improve low health literacy at a societal level through improved science education. In the US, the federal Department of Health and Human Services has released a “National Action Plan to Reduce Health Literacy” which is full of major policy ideas, but which will also take many years to succeed. National Action Plan to Improve Health Literacy.

Even the term “low health literacy,” on which much of the discussion of solutions is based, is problematic because it implicitly presupposes that the problem is solely with poor comprehension by patients who suffer from the “illness” of having poor health literacy, rather than a shared problem between health professionals and patients in which the shared goal of conveying crucial information from doctors to patients in a way that they properly comprehend is not being achieved.

What is needed is a simple, easy to use, inexpensive, and scalable solution that can help patients understand the information that is being conveyed to them quickly and accurately so that they can take the actions needed to protect and improve their health. Further what is needed are systems, devices and methods for reviewing and analyzing communications between a doctor and a patient that converts the communication from a specialized language to a broadly understood language.

Miscommunication between doctor and patient is not the only instance of barriers to proper care in the health care system. Patients also often struggle to understand the complex and specialized language of insurance plans. As a result, they may choose medical care that is sub-optimal and/or may make choices that result in serious and unexpected health costs. Over a third of Americans report having problems in understanding their health care coverage. Inability to understand health insurance coverage can have a negative impact on health care, including reduced use of preventive services.

A system that can provide simplified translations of medical coverage documents could improve consumer understanding of their care coverage and improve outcomes.

### SUMMARY

Disclosed are systems, devices and methods for reviewing and analyzing communications between a doctor or other source of health information conveyed in complex language and a patient, caregiver, or other recipient of such information that converts the communication from a specialized language to a broadly understood language, and enabling a social network based on shared conditions or interests.

A non-transitory computer readable medium can be provided. The non-transitory computer readable medium can have computer executable instructions, which when executed by a computer, cause the computer to execute operations comprising: retrieving a communication between a healthcare practitioner and at least one of a patient user and a patient caregiver user; analyzing the communication; generating a comprehension predictor model of the communication; predicting comprehension of the communication between the healthcare practitioner and at least one of the patient user and the patient caregiver user; translating the communication into a new communication; and delivering the new communication to at least one of the patient user and the patient caregiver user and enabling a social network based on shared conditions or interests.

Devices and one or more database servers may be implemented in a computer system using hardware, software, firmware, non-transitory computer readable media having instructions stored thereon, or a combination thereof and may be implemented in one or more computer systems or other processing systems. Hardware, software, or any combination thereof may embody modules and components used to implement the disclosed method.

One aspect of this disclosure provides a method for translating (e.g., interpretation) specialized language (e.g., language including medical terms) into broadly understood language (e.g., commonly understood language, plain language). The method includes obtaining a first input, which contains medical information related to a user, including one or more medical terms. The method then generates a first output based on this input using an algorithm. The first output provides an interpretation of the user's medical information by paraphrasing the medical terms into broadly understood language. The method includes delivering this first output to the user.

The disclosure may include some or all of the following optional features. The user's medical information may include a transcription of spoken words from a healthcare provider, the user's electronic health record (EHR), and/or a written communication from a healthcare provider. The output may provide to the user by displaying the first interpretation. Additionally, the output can be converted into a synthesized voice and played for the user. The method may use one or more algorithms, including natural language processing (NLP). The method can be also used to translate medical publications.

The algorithm can be trained through one or more iterations. Each iteration involves obtaining training input, which includes medical information. The algorithm then generates multiple training outputs based on this input. These outputs provide interpretations (e.g., translations) of the medical information, with one targeted at a first reading level and another at a second reading level. The training outputs are presented to the user, who provides feedback. The algorithm is adjusted based on this feedback. The medical information used for training can be the user's medical information. The first and second reading levels correspond to different Flesch Reading Ease Scores or Flesch-Kincaid Grade Levels. The feedback indicates the user's preference for a particular output, and the algorithm is refined to target that reading level.

Additionally, the method may generate suggested questions that provide additional valuable information for the user and incorporates them into the final output.

Another aspect of this disclosure provides a method for sharing a user's information. The method includes obtaining consent for data sharing from the user, de-identifying the user's data, transmitting the de-identified data to a server, and receiving a connection suggestion from the server. The data contains an interpretation (e.g., translation) of the medical information associated with the user, which includes one or more medical terms. This interpretation paraphrases the medical terms into language that is easy to understand.

The disclosure may include some or all of the following optional features. The consent for data sharing can be either explicit or implicit. The user's medical information may include an after-visit summary provided by a healthcare provider. The server may store the de-identified data and may allow access to this data for various partners, which may include biopharma companies, healthcare providers, payers, clinical trial sponsors, government health agencies, academic institutions, or patient advocacy organizations. The server may identify commonalities by analyzing non-medical keywords found in the user's data and the data of other users. Based on these commonalities, the server transmits connection suggestions.

Another aspect of this disclosure provides a method for sharing a user's information. The method involves obtaining first de-identified data from a first software application and second de-identified data from a second software application. It then stores both sets of data and identifies commonalities based on keywords found in each. If the first and second de-identified data share any commonalities, a connection offer is transmitted to both software applications.

The disclosure may include some or all of the following optional features. The keywords can be non-medical terms. The method may also include providing access to the first and second de-identified data to various partners. These partners may include biopharma companies, healthcare providers, payers, clinical trial sponsors, government health agencies, academic institutions, or patient advocacy organizations.

Another aspect of this disclosure provides a method for providing health insurance related answers. The method involves obtaining a question from the user about their health insurance plan. It then translates this question into language commonly used in the health insurance industry using an algorithm. Next, it analyzes the health insurance document related to the user's plan based on the translated question. The method provides an answer based on the results of this analysis.

The disclosure may include some or all of the following optional features. The question from the user can be an inquiry about health insurance coverage. Analyzing the health insurance document involves identifying relevant sections based on the translated question and then translating those sections into language that is easy to understand. The first answer provided includes these translated relevant sections.

The question from the user can be about health insurance coverage. Analyzing the health insurance document involves finding an answer from the health insurance document based on the translated question. This answer is then translated into easy-to-understand language. The answer provided to the user is based on this translated version.

### INCORPORATION BY REFERENCE

All publications, patents, and patent applications mentioned in this specification are herein incorporated by reference to the same extent as if each individual publication, patent, or patent application was specifically and individually indicated to be incorporated by reference.
Abridge, Stay on Top of Your Health, One Conversation at a Time (2023);
Amazon Web Services, Amazon Transcribe Medical Automatically Covert Medical Speech to Text (2023);
Augmedix, Ambient Medical Documentation (2023);
Butayeva, et al., The impact of health literacy interventions on glycemic control and self-management outcomes among type 2 diabetes mellitus: A systematic review. Journal of Diabetes. 2023; 15(9): 724-735. doi:10.1111/1753-0407.13436;
Deepscribe.AI, Reducing Physician Jargon with AI (2023);
Fireflies.AI, Automate your Meeting Notes (2023);
Gotlieb, et al. Accuracy in Patient Understanding of Common Medical Phrases. JAMA Netw Open. 2022; 5(11):e2242972;
Hickey et al., Low health literacy: Implications for managing cardiac patients in practice. Nurse Pract. August; 43(8):49-55 (2018); doi: 10.1097/01.NPR.0000541468.54290.49. PMID: 30028773; PMCID: PMC6391993;
HyperWrite, Technical Translator Simplify Technical Writing with AI (2023);
Jennings, This AI Startup Raised $15 Million to Help Patents Transcribe Doctor Appointments (Forbes, Oct. 6, 2020);
Medcorder, Keep Family in the Loop and Get Support (2023);
National Action Plan to Improve Health Literacy, U.S. Department of Health and Human Services, Office of Disease Prevention and Health Promotion, p. 1 (2010);
Olson et al., Communication Discrepancies Between Physicians and Hospitalized Patients. Arch Intern Med. 2010; 170(15):1302-1307. doi:10.1001/archinternmed.2010.239;
Richman, Online Tool Simplifies Complex Medical Language for Patients (NoteAid Sep. 14, 2017);
Roth, UPMC Helps Fund AI-Powered Patient Tool to Record, Transcribe Medical Visits and Drive Patient Engagement (Health Leaders Medica Jul. 14, 2020);
Tongue, et al., Communication Skills for Patient-Centered Care: Research-Based, Easily Learned Techniques for Medical Interviews That Benefit Orthopaedic Surgeons and Their Patients. The Journal of Bone & Joint Surgery 87(3):p 652-658, March 2005;
Zaidman, et al. Impact of parental health literacy on the health outcomes of children with chronic disease globally: A systematic review;
U.S. Pat. No. 11,094,322 issued Aug. 17, 2021 to Lavery, et al.;
U.S. Pat. No. 11,650,732 issued May 16, 2023 to Strader, et al.;
U.S. Pat. No. 11,670,408 issued Jun. 6, 2023 to Pinto;
U.S. Pub 2012/0101847 A1 published Apr. 26, 2012 to Johnson, et al.;
U.S. Pub 2018/0197548 A1 published Jul. 12, 2018 to Palakodety et al.;
U.S. Pub 2020/0058400 A1 published Feb. 20, 2020 to Leonard;
U.S. Pub 2022/0115134 A1 published Apr. 14, 2022 to Strader et al.;
U.S. Pub 2022/0375605 A1 published Nov. 24, 2022 to Lipton et al.; and
U.S. Pub 2023/0055094 A1 published Feb. 23, 2023 to Co et al.

### BRIEF DESCRIPTION OF THE DRAWINGS

The novel features of the disclosure are set forth with particularity in the appended claims. As will be appreciated by those skilled in the art, the various features illustrated in the drawings may not be drawn to scale. Moreover, the illustrations presented in the present disclosure are not meant to be actual views of any particular apparatus (e.g., device, system, etc.) or method, but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

A better understanding of the features and advantages of the present disclosure will be obtained by reference to the following detailed description that sets forth illustrative embodiments, in which the principles of the disclosure are utilized, and the accompanying drawings of which:

FIG. 1 is a diagram of a system to translate (e.g., analyze and covert) specialized language to broadly understood language;

FIG. 2 is a diagram of a process flow for spoken words;

FIG. 3 is a diagram of a process flow for written words;

FIG. 4 is a diagram of a process flow for electronic records;

FIG. 5 is a diagram of a system facilitating user connections using an application;

FIG. 6 is a diagram of a network of users using an application;

FIG. 7 is a diagram of a system connecting application and partners;

FIG. 8 is a flowchart of an example arrangement of operations for a method of providing broadly understood output tailored to a specific reading level;

FIG. 9 is a flowchart of an example arrangement of operations for a method of providing broadly understood output tailored to a specific reading level;

FIG. 10 is a flowchart of an example arrangement of operations for a method of providing broadly understood output tailored to a specific reading level;

FIG. 11 is a flowchart of an example arrangement of operations for a method of providing broadly understood output tailored to a specific reading level;

FIG. 12 is a flowchart of an example arrangement of operations for a method of establishing patient networks;

FIG. 13 is a flowchart of an example arrangement of operations for a method of sharing user's data (e.g., translated health record) with one or more partners;

FIG. 14 is a diagram of system, which is operable to answer the user's questions related to their health insurance plan by analyzing the user's health insurance plan in accordance with some implementations of the disclosure;

FIG. 15 is a flowchart of an example arrangement of operations for a method of providing answers to a user's health insurance plan related questions in accordance with some implementations of the disclosure; and

FIG. 16 illustrates a diagrammatic representation of a machine in the example form of a computing device.

### DETAILED DESCRIPTION

The devices, systems and methods describe are operable to assist the millions of Americans and people around the world who miss or misinterpret vital health information during communications with healthcare providers because the patient misunderstands the specialized language (e.g., medical terminology) used by healthcare providers. One component of the system may include a software application that may be utilized by smartphones, wearables, desktop and laptop computers, and other hardware platforms. The software application can be a mobile application or a web application. The software can be native to a device or downloadable. The software application may be operable to translate (e.g., paraphrase) medical information, such as recordings of doctor visits, messages from healthcare professionals, and/or materials provided by healthcare providers from specialized language (that may be difficult for a non-specialist to understand) into broadly understood language that may be used by patients every day. Such a translation may improve understanding by patients of the medical information received and may improve health outcomes. The system may use natural language processing (NLP) with reference to publicly available lexicons and other sources that translate medical jargon (e.g., the specialized language, medical terminology) into more broadly understood used phrasing. In some implementations, NLP is trained with proprietary databases and corpora of information generated for training purposes. This enables the translation of both spoken and written text from the specialized language into common language. The system may allow either healthcare users or patient users to set and/or adjust an algorithm associated with the system to translate the specialized language into the broadly understood language, at the preferred reading level of the user.

Artificial intelligence (AI) can be used to enable the devices and systems to learn from user responses and reactions to previously provided translations.

The system may enable patient users to opt to share de-identified information with other patient users of the system, and/or with partner organizations. The de-identified data are records that have a re-identification code and enough personally identifiable information removed or obscured so that the remaining information does not identify an individual and there is no basis to believe that the available information can be used to specifically identify any one individual. By sharing de-identified information with other patient users of the system, a patient user can connect with another patient user with a similar medical condition and/or who may be receiving a similar treatment without fear of having their identity revealed. Further, patient users need not rely on complex medical terminology to find similarly situated patient users. Instead, the patient users may use broadly understood language (e.g., commonly understood language), which may potentially facilitate wider and/or more accurate connections. Using the system, the patient users may be able to share text, video, audio, and/or direct messages with either a set of other patient users, or with individual users (such as patient caregiver users), creating a community of interest focused on a similar condition or treatment protocol.

The data sharing agreement through which a patient user opts to share de-identified data with other patient users may also include permission to share the de-identified data with partner organizations. The partner organizations could include biopharmaceutical companies seeking real world evidence about the use and efficacy of products, clinical trial sponsors trying to recruit participants, payers trying to optimize utilization, nonprofit advocacy groups, and/or many others. The system may be operable to serve advertising to a patient user that is tailored to the patient user's medical information.

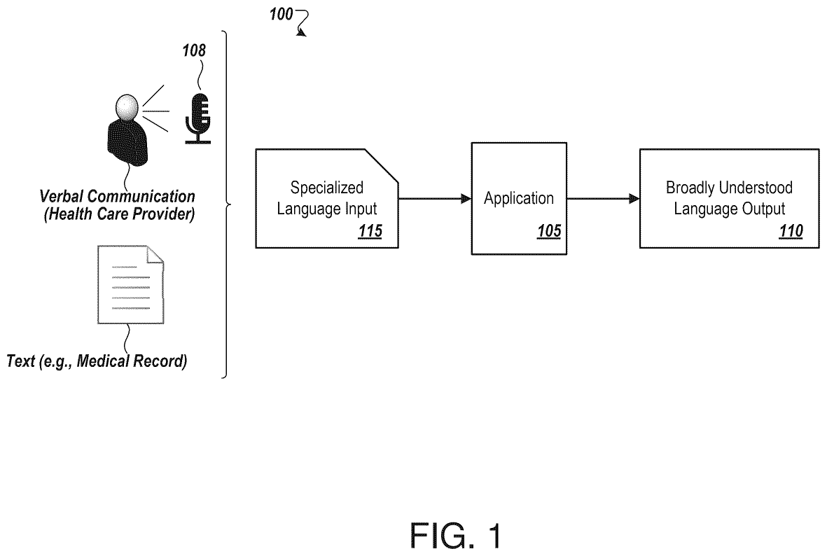

FIG. 1 is a diagram of system 100 operable to translate (e.g., analyze and convert) a specialized language in a communication to a broadly understood language (e.g., plain language, commonly understood language) in accordance some implementations with the disclosure. In other words, the system 100 is configured to simplify complex medical terms into plain language suitable for non-medical users (e.g., patient user, patient caregiver). System 100 may include a software application 105 configured to obtain specialized language input 115 and translate it into broadly understood language output 110 (also referred to as common language output or plain language output in this disclosure).

Software application 105 may be implemented on smartphones, wearables, desktop computers, laptop computers, and/or other computing hardware platforms. System 100 may also be operable to perform one or more operations, including: translating text and/or spoken words from a specialized language into a broadly understood language; adjusting (e.g., tuning) the algorithm (e.g., analysis algorithm) associated with the software application 105 so that the software application 105 is able to generate the broadly understood language output 110 at a target reading level; connecting users of the software application 105; and/or sharing data with one or more partners (e.g., biopharma companies, healthcare providers, payers, clinical trial sponsors, government health agencies, academic institutions, patient advocacy organizations, public health agencies).

System 100 may use a recording device 108 to obtain the specialized language 115 in a communication. Recording device 108 may be a peripheral recording device that is separate from system 100. Additionally, the recording 108 device may be connected to the system 100, incorporated into the system 100 and/or integral with the system 100. Software application 105 may use the recording device 108 to record the specialized language 115 and/or the software application 105 may perform an analysis on the specialized language 115. Alternatively, or additionally, the software application 105 may be operable to obtain, store, and/or analyze text (e.g., medical record, medical paper) from other sources, such as from a user/patient, from a healthcare provider, and/or from other sources.

System 100 (and/or components included in the system 100, such as the recording device 108) may record a communication occurring during a medical visit, and the software application 105 may use natural language processing (NLP) to translate the specialized language 115 used by doctors and/or healthcare providers into a broadly understood language output 110. As such, the system 100 may provide a patient a permanent record of the medical visit in the broadly understood language optimized for the patient's comprehension.

System 100 may be operable to adjust (e.g., tune) the algorithm (e.g., analysis algorithm) associated with the software application 105 to a preferred level of language complexity. For example, the Flesch Reading Ease Score may be used as a metric to measure how difficult a given passage of text is to read, using a scale of 0 to 100, with 100 being the easiest to read. Different score ranges correspond to different grade levels of reading ability, according to the related Flesch-Kincaid Grade Level.

Scores from 0 to 30 may represent the least easy language to read. A communication translated to text in this score range may be accessible to only patients with an advanced education level. Much of the specialized language 115 may be in this range. The system 100 may allow patient users to set a preferred complexity range, such as at a moderate complexity (e.g., corresponding to a Flesch Reading Ease score of about 50, representing a high school graduate), or at a low complexity range (corresponding to a Flesch Reading Ease score of about 70, corresponding to a seventh grade reading level). The adjusting (e.g., tuning) of the algorithm may also be operable to adjust the broadly understood language output 110 from the software application 105 to match the patient user's comprehension level.

System 100 may be operable in one or more languages, which may be determined by the user of the system 100. For example, the system 100 may use English, Spanish, French, etc. System 100 may be operable to include more gradations of reading complexity than listed, which may allow the user to more finely tune the broadly understood language output 110 to a desired level (e.g., target reading level).

In addition to considering education level and language, the system is operable to account the education level of a patient in combination with a native language for the patient. Thus, for example, the language used for a native English speaker with a high school education might be different than a native Chinese speaker with a high school education. Similarly, the language used for a native English speaker with a high school education might be different than a native Chinese speaker with a PhD, or a native Chinese speaker with a PhD that has lived in in English speaking country for 10 years. In some configurations, the broadly understood language for a Chinese speaker with a PhD could be translated into similarly complex language in Chinese or simpler terminology in English.

The software application 105 may allow one or more patient users to share data with one or more other patient users. When patient users decide to share data, de-identified data associated with them may be available for connecting with other users of the system 100 (e.g., other users of application 105 on their smartphones) and/or for sharing with one or more partner organizations (as described below).

Software application 105 may be operable to perform an analysis of the user data included in the account of each user that has opted to share data. Alternatively, or additionally, the software application 105 may be able to detect instances where two or more different users may have similar clinical descriptions of conditions or similar treatment patterns. In instances in which similarities are detected, the software application 105 may inform the users having similar conditions of a possible match to another user with a similar profile. Each user will then be able to accept any other such user into their network, in a manner similar to other social networks.

If a patient opts in, a patient users may be allowed to share text, video, photographs, and/or other electronic information with other patient users in their network, either individually or across an entire group. Through establishing a platform of users with similar clinical profiles, the system 100 may create a linked community of patients able to directly connect and/or share experiences.

Thus, in an example, when a user opts in and/or does not opt out, the user can share de-identified data with other users and partners in the system as a general setting. Once a choice to share information is provided, if a patient matches with other users (other patients, caregivers, healthcare providers etc.), the user is free to send whatever they like via the network. The information can be de-identified data and/or identifiable personal information related to or unrelated to a clinical condition in the form of text, audio, video or other digital format.

Users interested in network access but who do not themselves have a clinical history of a particular disorder (such as a caregiver, family member or other user) may establish a guest account, and the guest user may request to join one or more networks. Users in the one or more networks may choose to connect with any other accounts, guest or otherwise. Any user can determine which other users a user wants to connect or disconnect with.

System 100 may be operable to share de-identified patient user data with the one or more partners associated with the system 100, as the patient users opt-in to/do not opt out of data sharing operations. The one or more partners may include, but are not limited to, pharmaceutical companies that use the data for real world evidence on the performance of company products, sponsors of clinical trials that are seeking to enroll patients with particular clinical characteristics, nonprofit patient organizations that are seeking to build membership, providers seeking to optimize treatment protocols, and/or health care payers.

Software application 105 may include a capacity to accept information from the partners, which may be in the form of digital advertising, which may be targeted to patient users having specific clinical characteristics. Software application 105 may include a capacity to require a separate opt-in for patient users who wish to receive these communications.

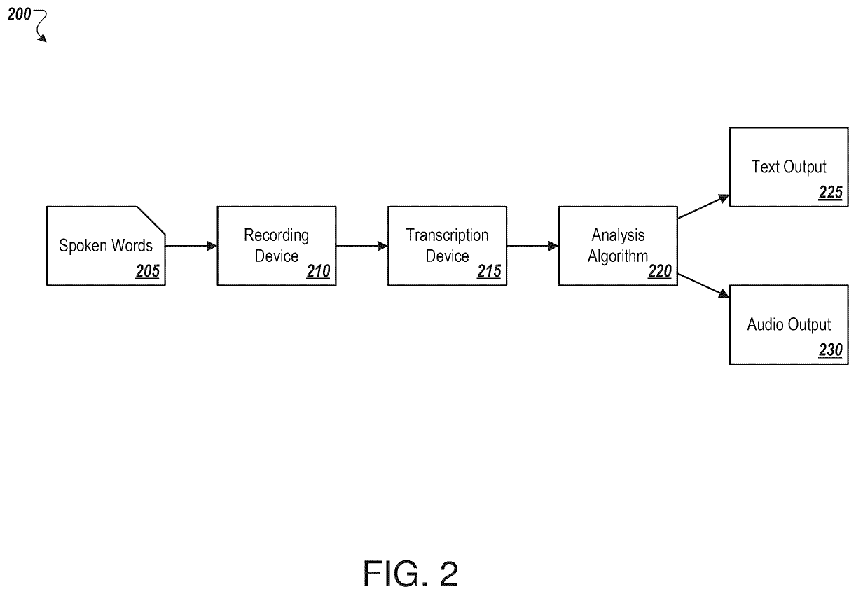

FIG. 2 is a diagram of a process flow 200 for spoken words, in accordance with at least one embodiment of the present disclosure. The process flow 200 may begin as a medical professional or healthcare provider speaks or communicates, generating spoken words 205. Spoken words 205 may be captured by a recording device 210. Recording device 210 may be an intrinsic part of the hardware platform on which an application resides (e.g., the software application 105 of FIG. 1), such as in the case of a smartphone with a built-in microphone. The recording device 210 may also be an external peripheral device, such as in the case of an external recording device used as part of a desktop computer system.

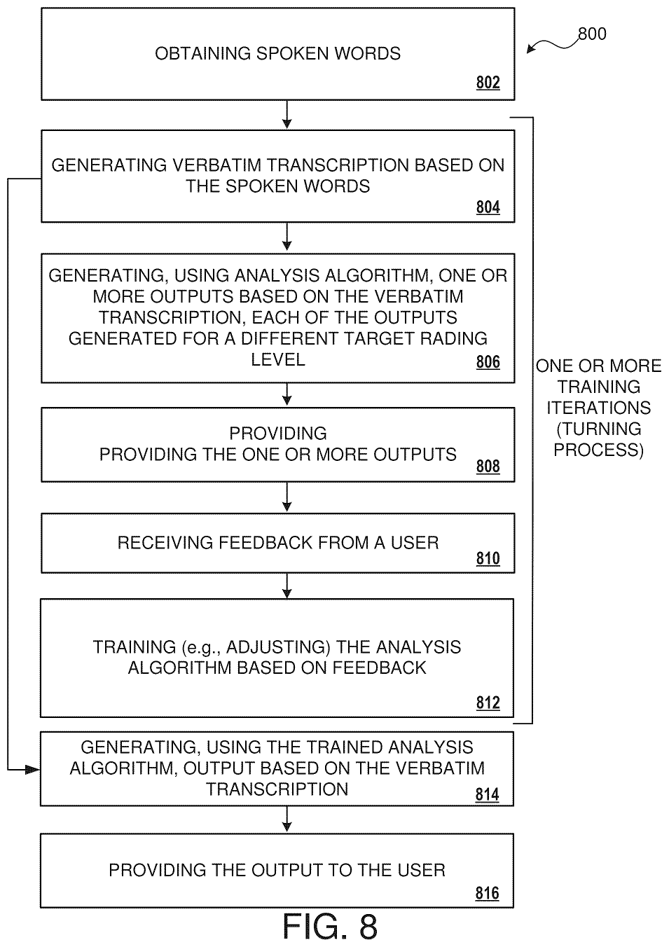

Recording device 210 may relay the spoken words 205 to a transcription device 215 that may be part of the software application. The transcription device 215 may prepare a verbatim transcription of the spoken words 205, which may constitute the base material on which an analysis by an analysis algorithm 220 may be performed. The data from the verbatim transcription may be passed to the analysis algorithm 220, which may be located within the software application. The analysis algorithm 220 may be operable to translate the verbatim transcription to a broadly understood language (e.g., plain language), which may be based on the preferences of the user. The preferences of the patient user may be defined and/or implemented as part of the analysis algorithm 220. The output of the analysis algorithm 220 may be displayed to the patient user or caregiver user either on as a text output 225 (e.g., a digital display of text) and/or audibly as an audio output 230, such as via a voice synthesizer. As will be described in FIG. 8-FIG. 11, in some configurations, the output of the analysis algorithm 220 (e.g., text output 225, audio output 230) may include suggested questions.

In some configurations, users can provide feedback to indicate how well they understand a particular communication and/or request an increase or decrease if the complexity of the language of the communication. The feedback can be used to further train the algorithm.

Training the analysis algorithm 220 may utilize a metric associated with the Flesch Reading Ease Score, which was developed in the 1940's to measure how simple or difficult a passage of text is to read. The metric measures the length and complexity of sentences, as well as the length and complexity of words within the sentences.

The Flesch Reading Ease Score is widely used in marketing, the military, and in many contexts in which easy readability is valued. For example, under Virginia state law, all insurance policy documents sent to the public must reach a legally specified threshold on the Flesch Reading Ease Score in order to be acceptable. The Flesch Reading Ease metric scores text on a scale of 0 to 100, with 100 being the easiest to read. The Flesch Reading Ease Score is often paired with the Flesch-Kincaid Grade Level, which provides an index of the grade level of education (based on US schools) needed to read text at different points along the Flesch Reading Ease Score. For example, a Flesch Reading Ease score of between 70 and 80 would yield a Flesch-Kincaid Grade Level of between 7^{th} and 8^{th} grade, while any text scoring at 30 or below would require a college degree or beyond to be comprehensible.

The software application may allow patient users to select a preferred level of reading, based on the Flesch Reading Ease Score. Alternatively, the software application may allow a healthcare provider to set an initial reading level which can then be modified by the patient in response to translated communications received by the system.

The starting point for many communications by health professionals (e.g., the specialized language) may be language that may be a very low level of readability for most patients, who lack specialized medical training, due to its intrinsic complexity. Alternatively, or additionally, the specialized language may be compounded by the heavy use of specialist jargon. For example, the following statement: “While in a minority of cases, MD or ataxia were a symptom of known clinical entities, in most children, the etiology was suspected to be of autoimmune origin without further assigned diagnosis. Children either presented with ataxia, but different from the well-known postinfectious acute cerebellar ataxia, or had hypo-/hyperkinetic MD, which were choreatic in most cases” has a Flesch Readability Ease Score of 8.7, equating to a grade level of 19.1—in other words, likely requiring post-graduate education to understand. The software application may provide patient users with a translation of such text at different Flesch Reading Ease levels and may allow patient users to select their preferred level. The selected readability level is the level of reading ease at which the software application may provide translated text for the patient user.

For example, the above statement can be translated to a moderate complexity (e.g., 47.8 metric for a Flesch-Kincaid Grade level of 12.4, or a high school graduate) and a low complexity (e.g., 72.6 metric, for a Flesch-Kincaid Grade level of 7^{th} Grade), respectively as follows:
“Less than half the time, movement disorders or uncoordinated movement were a symptom of known diseases. In most children, doctors thought that the cause of disease was an autoimmune condition that they didn't recognize. Children came to the doctor's office with uncoordinated movement problems that were different from similar diseases, or the children had movement disorders where they moved too much or too little, and often had slow rhythmic movements.”
“Less than half the time, when people had problems moving, it was a symptom of known diseases. Doctors thought the children had a disease that they didn't recognize where the body reacted to itself. Children came to the doctor's office with problems with the way they moved or the children had problems where they moved too much or too little. These problems were different from similar diseases. They often moved slowly and with movements that looked like dances.”

By allowing the patient user to set the analysis algorithm 220 at their desired reading level (e.g., training the analysis algorithm 220 to target a specific reading level), the software application may deliver text to the user in the format in which it will be maximally accessible to the patient user.

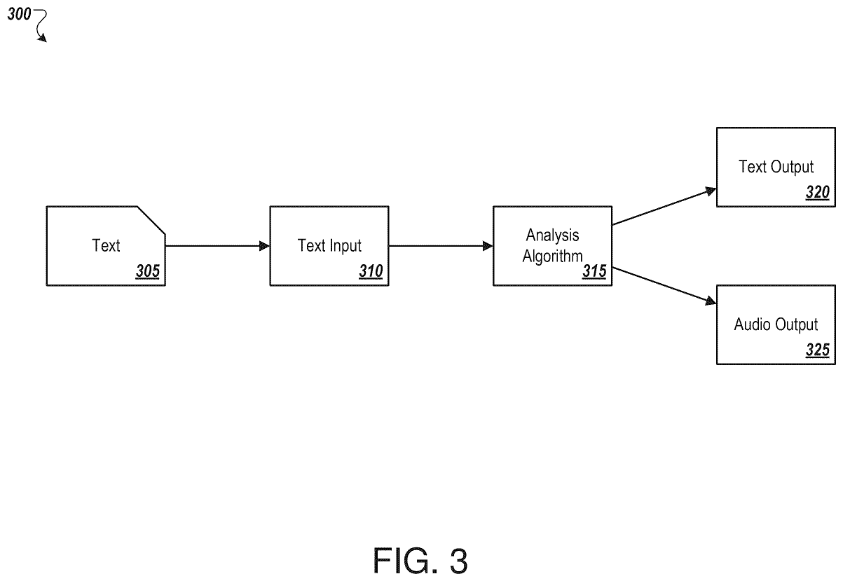

FIG. 3 is a diagram of a process flow 300 for written words, in accordance with at least one embodiment of the present disclosure. The input to a software application (e.g., the software application 105 of FIG. 1) may be provided in as text 305. For example, the text 305 may include text from a medical chart, text from an email, text, and/or other communication from a medical professional, external information, such as a medical publication, or health insurance plan (e.g., user's health insurance policy document). The text 305 may be directly captured by a system (e.g., application server associated with the software application) running the software application and/or the text 305 directly entered by a healthcare user or a patient user as text input 310.

Text 305 and/or the text input 310 may be obtained by an analysis algorithm 315 within the software application. The analysis algorithm 315 may translate (e.g., analyze and covert) the text 305 and/or the text input 310 transcript to a broadly understood language, based on the preferences of the patient user (e.g., based on a target reading level) that may be defined within the analysis algorithm 315. The output of the analysis algorithm 315 may be displayed to the patient user either on as a text output 320 (e.g., a digital display of text) and/or audibly as an audio output 325, such as via a voice synthesizer. As will be described in FIG. 8-FIG. 11, in some configurations, the output of the analysis algorithm 315 (e.g., text output 320, audio output 325) may include suggested questions.

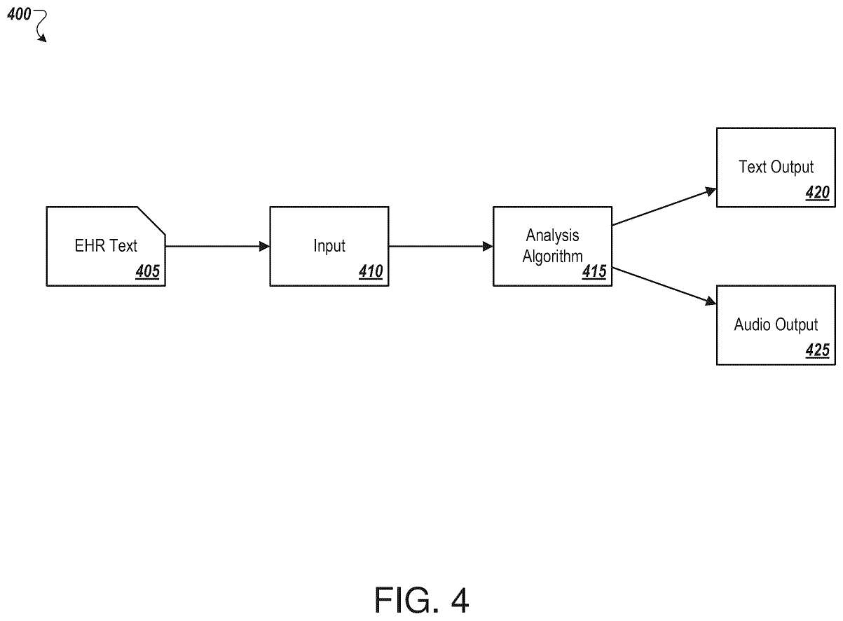

FIG. 4 is a diagram of a process flow 400 for electronic records, in accordance with at least one embodiment of the present disclosure. The input to an application (e.g., the software application 105 of FIG. 1) may be provided as electronic health record (EHR) text 405. EHR text 405 may be an input 410 into the software application. EHR text 405 and/or the input 410 may be obtained by an analysis algorithm 415 within the software application. The analysis algorithm 315 may translate (e.g., analyze and covert) the EHR text 405 and/or the input 410 to a broadly understood language, based on the preferences of the patient user (e.g., based on a target reading level) that may be defined within the analysis algorithm 415. The output of the analysis algorithm 415 may be displayed to the patient user either on as a text output 420 (e.g., a digital display of text) and/or audibly as an audio output 425, such as via a voice synthesizer. As will be described in FIG. 8-FIG. 11, in some configurations, the output of the analysis algorithm 415 (e.g., text output 420, audio output 425) may include suggested questions.

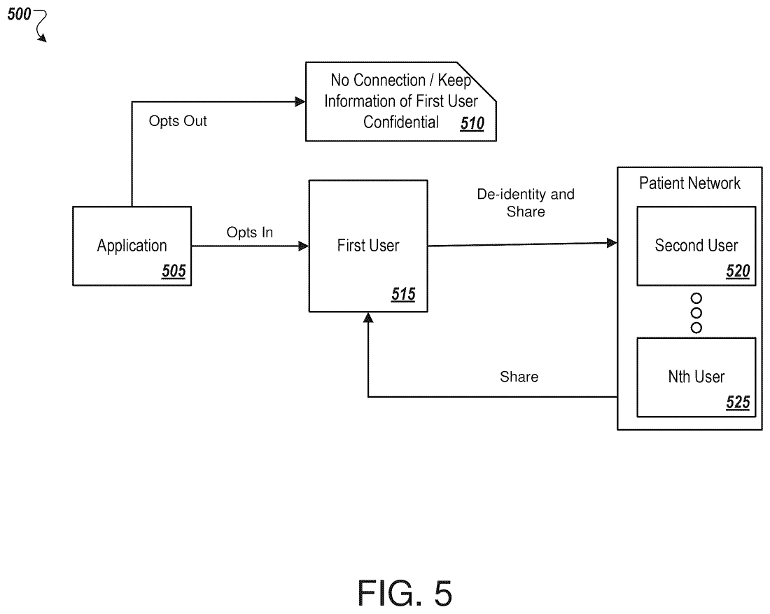

FIG. 5 is a diagram of a system 500 facilitating user connections using a software application 505, in accordance with at least one embodiment of the present disclosure. The software application 505 may allow users to opt in to data sharing. Alternatively, or additionally, the software application 505 may set data sharing as a default from which users can opt out.

In instances in which a user opts out of data sharing using the software application 505, the patient user may no longer be connected and/or may not experience any of the network functionality, as described herein. Alternatively, if a first user 515 opts in to data sharing (either by actively opting in (e.g., giving an explicit sharing permission) or not opting out (e.g., giving an implicit sharing permission), depending on the configuration of the software application 505), then de-identified information about the first user 515 may be shared for a variety of uses. Each user may have the option to establish a public-facing user name which need not reflect their own.

One major such use is to enable users (e.g., the first user 515, a second user 520, and/or an nth user 525) to identify other users who share similar health conditions, are receiving similar treatments, or otherwise have similar interests. The software application 505 may include the capability to analyze key words in the shared data that each user has analyzed using the software application 505.

As illustrated, the first user 515 has opted in to the data sharing option associated with the software application 505. The text that the first user 515 has analyzed with the software application 505 may be compared with that of the second user 520 and/or the nth user 525 to determine if the text from any of the other users in the patient network matches key words in the text that the first user 515 has run through the software application 505.

In instances in which the second user 520 has analyzed text using the software application 505 that has similar key words to those of the first user 515, the software application 505 may offer the first user 515 and the second user 520 an option to establish a 1:1 connection. Alternatively, or additionally, the software application 505 may offer the first user 515 to be added to any user connections that the second user 520 has already established, and for the second user 520 to be added to any user connections that the first user 515 has already established. As such, the software application 505 may facilitate the formation of patient networks that may be linked by broadly understood medical conditions, treatments, or interests.

In some embodiments, upon “explicit” or “implicit” sharing permission from the first user 505, the software application 505 (e.g., software application on the first user smartphone) de-identifies the data of the first user 505 (e.g., de-identified broadly understood language) and transmits (e.g., upload) the data to the software application server. Similarly, software application 505 associated with the second user 520 transmits the data of the second user 520 to the application server, and software application 505 associated with the nth user 525 transmits the data of the nth user 525 to the application server. The application server may store all the shared data in a database (e.g., database server).

The application server may analyze key words in the shared data (e.g., shared data stored in the database) stored in the database and determine commonalities in the key words between users.

In instances in which the first user 515 and the second user 520 have commonalities in the key words, the application server may offer the first user 515 and the second user 520 an option to establish a 1:1 connection. Alternatively, or additionally, the application server may offer the first user 515 to be added to any user connections that the second user 520 has already established, and for the second user 520 to be added to any user connections that the first user 515 has already established. As such, the application server may facilitate the formation of patient networks that may be linked by broadly understood medical conditions, treatments, or interests.

Each user 515, 520, and 525 can analyze key words in the shared data (e.g., performing keyword searches using non-medical terms) stored in the database and determine commonalities in the key words between users. For example, the first user 515 analyze key words in the shared data (e.g., shared data stored in the database) stored in the database and determine commonalities in the key words between the second user 520 and nth user 525 using the software application 505 on own device. In instances in which the first user 515 and the second user 520 have commonalities in the key words, the software application 505 of the first user 515 may offer the first user 515 and the second user 520 an option to establish a 1:1 connection. Alternatively, or additionally, the software application 505 of the first user 515 may offer the first user 515 to be added to any user connections that the second user 520 has already established, and for the second user 520 to be added to any user connections that the first user 515 has already established. As such, the application software 505 may facilitate the formation of patient networks that may be linked by broadly understood medical conditions, treatments, or interests.

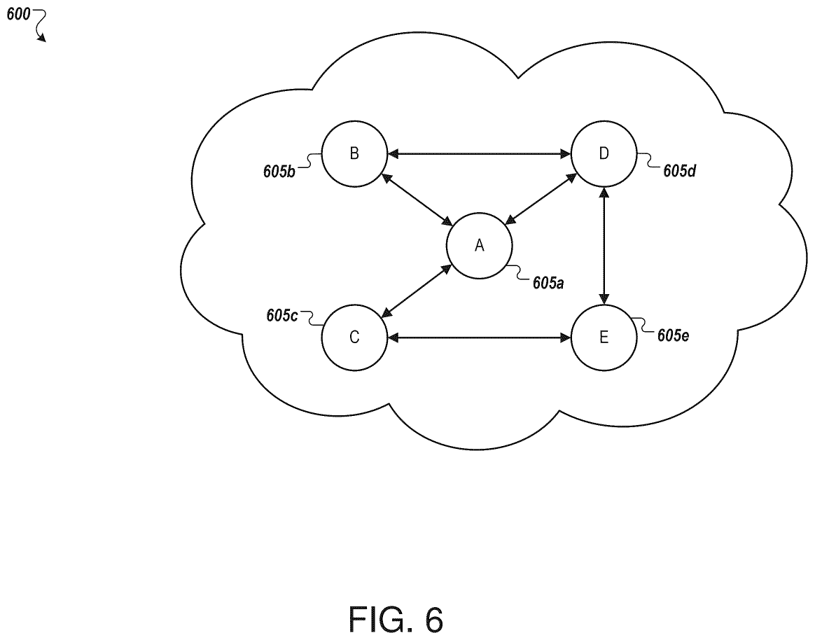

FIG. 6 is a diagram of a network 600 of each of users 605 using a software application, in accordance with at least one embodiment of the disclosure. The users 605 include patient users, healthcare users, caregiver users, family member users, moderator users, etc. In the network 600, user A 605a may be connected with one or more of the other users 605, the connection illustrated by the line between the users 605. In some instances, the user A 605a may share key words in broadly understood language with one or more of the users 605 (with which the user A 605a may not yet be connected with, such as user E 605e). As described relative to FIG. 5, each of the users 605 may have allowed their data to be shared. The network 600 (e.g., network 600 including a software application server associated with the software application used by the users 605) may have determined commonalities in the key words that each of the users 605 has analyzed through a software application (e.g., the software application 105 of FIG. 1) and (based on the commonalities) the software application server may have offered the users an opportunity to be connected to other users sharing those commonalities in the network 600. As such, each of the users 605 that are connected in the network 600 has agreed to connect, and thus the network 600 has formed among the users 605, where any one of the existing users 605 may be removed and/or additional users may be added to the network 600.

The users 605 of the network 600 may share information with one another, either individually/directly, and/or across the network 600. The shared information may take whatever digital form the users 605 prefer, including text, visual images, and/or video. Each of the users 605 may de-link from any other user in the network 600, so that if a particular user is sharing unwanted content, that user will quickly become disengaged from the network 600. The users 605 who are not themselves patients will have the option of entering specific key words into the system to trigger the system to search for other users sharing those key words. The non-patient user would then have the opportunity to link with any other user using those key words, notwithstanding the fact that the non-patient user is not experiencing a condition associated with those key words. As with patients who are users, any user receiving a connection request from a non-patient user would have the option to accept or decline a connection request.

Public facing usernames may include a notation as to whether the user is experiencing a condition themselves, a family member of a person experiencing such a condition, and/or a user who is otherwise interested in the condition. Such users might include public health researchers, members of advocacy groups, and/or medical professionals.

The network 600 (and/or other networks) established among the users 605 of the software application may become a platform for the users 605 to share blogs, observations about specific conditions, and/or sources of information with the other users 605 experiencing or otherwise interested in that condition, thus creating a community of interest based on a shared clinical condition or a shared interest in a particular clinical condition.

The software application may be operable to receive advertising, which may be targeted to a specific medical condition based on the data shared by the users 605. One configuration of the software application may disallow such advertising. Another configuration of the software application may allow such advertising, but may be limited to the users 605 who have opted in to receiving it.

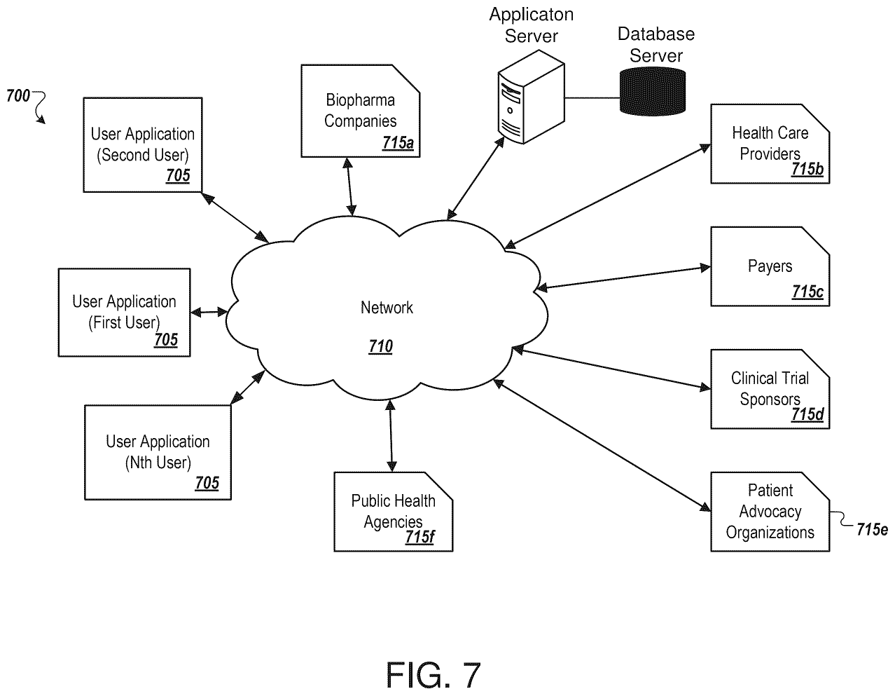

FIG. 7 is a diagram of a system 700 connecting an application 705 and partners 715, in accordance with at least one embodiment of the present disclosure. When a user allows his or her de-identified data to be shared with the network 710, the terms of use will specify that the user data may be shared with the partners 715. The partners 715 may include one or more of the following:
Biopharma companies 715a—who may use the user data for purposes that may include but are not limited to improving their understanding of the performance of their products through real world evidence
Healthcare providers 715b—who may use the user data for purposes that may include but are not limited to improving their provision of care or track outcomes
Payers 715c—who may use the user data for purposes that may include but are not limited to care management
Clinical trial sponsors 715d—who may use the user data for purposes that may include but are not limited to improving recruitment of participants in clinical trials
Patient advocacy organizations 715e—who may use the user data for purposes that may include but are not limited to improving better the needs of their constituents
Public health agencies 715f—who may use the user data for purposes that may include but are not limited to improving understand how best to manage or prevent illness, or to track outbreaks of disease

System 700 may include an application server. The application server may be realized through various web-based technologies, such as, but not limited to, a Java web-framework, a .NET framework, a PHP framework, or any other web-application framework. Examples of the application server may include, but are not limited to, computers, laptops, mini-computers, mainframe computers, mobile phones, tablets, and any non-transient and tangible machines that may execute a machine-readable code, a cloud-based server, or a network of computer systems.

System 700 can also include a database server that includes suitable logic, circuitry, interfaces, and/or code, executable by the circuitry, that may be configured to perform one or more operations for managing and storing various forms of data. The database server may be configured to store data, such as the test data retrieved from any of the users of the system. The database server may be configured to receive a query from the application server to extract the data stored in the database server. Based on the received query, the database server may be configured to provide the requested data to the application server over the communication network. Examples of the database servers may include, but are not limited to, the open-source relational database management system MySQL® developed by Oracle®.

FIG. 8 is a flowchart of an example arrangement of operations for a method 800 of providing broadly understood output tailored to a specific reading level. The method 800 may be performed by processing logic that may include hardware (circuitry, dedicated logic, data processing hardware, etc.), software (such as is run on a general purpose computer system or a dedicated machine), or a combination of both, which processing logic may be included in any computer system or device. For simplicity of explanation, methods described herein are depicted and described as a series of acts. However, acts in accordance with this disclosure may occur in various orders and/or concurrently, and with other acts not presented and described herein. Further, not all illustrated acts may be used to implement the methods in accordance with the disclosed subject matter. In addition, those skilled in the art will understand and appreciate that the methods may alternatively be represented as a series of interrelated states via a state diagram or events. Additionally, the methods disclosed in this specification are capable of being stored on an article of manufacture, such as a non-transitory computer-readable medium, to facilitate transporting and transferring such methods to computing devices. The term article of manufacture, as used herein, is intended to encompass a computer program accessible from any computer-readable device or storage media. Although illustrated as discrete blocks, various blocks may be divided into additional blocks, combined into fewer blocks, or eliminated, depending on the desired implementation.

The method 800, at operation 802, includes obtaining (e.g., receiving), by a data processing hardware, spoken words (e.g., spoken words from a healthcare provider). As discussed, these spoken words (e.g., specialized language) may include medical terminology that is difficult for a patient to understand.

The method 800, at operation 804, during one or more training iterations, includes generating, by the data processing hardware, a verbatim transcription based on the spoken words. Natural language processing (NLP) may be implemented for generating the verbatim transcription from the spoken words.

The method 800, at operation 806, during the one or more training iterations, includes generating, by the data processing hardware, one or more outputs (e.g., translations, interpretation) using analysis algorithm. As discussed, the analysis algorithm may be operable to translate (e.g., paraphrase) the verbatim transcription to a broadly understood language. For example, the software application may translate the verbatim transcription to a broadly understood language targeting a specific reading level (e.g., reading level based on Flesch-Kincaid Grade level, reading level based on Flesch Reading Ease Score, reading level based on education level). In another example, the software application may translate (e.g., paraphrase) the verbatim transcription into multiple translations (e.g., interpretations), each translation tailored to a specific reading level (e.g., first translation aimed at a middle school graduate level and second translation aimed at a high school graduate level).

The method 800, at operation 808, during the one or more training iterations, includes providing, by the data processing hardware, the one or more outputs (e.g., translations) to a user (e.g., patient user, caregiver user). As discussed, the output of the analysis algorithm may be displayed to the user either on as a text output (e.g., a digital display of text) and/or audibly as an audio output, such as via a voice synthesizer.

The method 800, at operation 810, during the one or more training iterations, includes receiving, by the data processing hardware, feedback from the user. For example, the user may provide feedback indicating whether the output (e.g., translation) is too difficult or too easy to read. In another example, the user may indicate which of the multiple outputs (e.g., translations) is most suitable for reading.

The method 800, at operation 812, during the one or more training iterations, includes training (e.g., adjusting, turning), by the data processing hardware, the analysis algorithm based on the feedback received from the user. For example, when the user indicates that the output targeted at the high school graduate reading level is too difficult to read, the analysis algorithm is adjusted (e.g., trained) to target a lower reading level (e.g., middle school graduate reading level). Similarly, when the user indicates that the output targeting the high school graduate reading level is too easy to read, the analysis algorithm is adjusted (e.g., trained) to target a higher reading level (e.g., college graduate reading level). In another example, when the user indicates which of the multiple outputs is most suitable for reading, the analysis algorithm is adjusted (e.g., trained) to target the reading level corresponding to the preferred output (e.g., indicated translation).

The method 800, at operation 814, for subsequent translations, includes generating, by the data processing hardware, output (translation) using the trained analysis algorithm (e.g., adjusted analysis algorithm) based on subsequent verbatim transcriptions.

The method 800, at operation 816, includes providing, by the data processing hardware, the output to the user (e.g., patient user, caregiver user). As discussed, the output of the analysis algorithm may be displayed to the user either on as a text output (e.g., a digital display of text) and/or audibly as an audio output, such as via a voice synthesizer. The output may include a plain language summary of a doctor's visit (e.g., after-visit summary) tailored to the adjusted target reading level. Additionally, the output may provide simplified language translations of health insurance plan documents and/or other related information to allow the user to better understand the existence, extent and/or conditions of coverage of health care services under that health insurance plan.

Additionally, the output may include one or more suggested questions for the user to ask their healthcare provider, based on the user's condition as described in the output and/or the verbatim transcription.

The method 800 further includes generating, by the data processing hardware, one or more suggested questions (that were not addressed in the output and/or verbatim transcription) for the user to ask their healthcare provider. These suggested questions are then provided alongside the output, creating a comprehensive communication tool for the user. For example, this operation can be performed between operation 814 and operation 816.

For example, based on the output and/or verbatim transcription, the analysis algorithm may generate one or more suggested questions for the user to ask the healthcare provider that may provide additional valuable information for the user. These questions may include: “How will I know if my infection is getting worse?”, “Are there foods that I shouldn't eat with medicines you are prescribing?”, or “Are there any activities I should avoid for a faster recovery?”

Once these suggested questions are provided to the user, they have the opportunity to follow up with their healthcare provider for further clarification or guidance. This follow-up may take various forms, such as scheduling an in-person appointment, utilizing telehealth services, or communicating through secure messaging platforms that many healthcare systems offer. Such flexibility in communication channels allows the user to choose the most convenient option based on their circumstances and preferences.

In this follow-up interaction, the user can present the suggested questions to their healthcare provider, ensuring they receive comprehensive answers tailored to their individual situation. For instance, the healthcare provider can explain the signs of worsening infection in detail, provide dietary recommendations that promote healing, and clarify which activities should be limited to facilitate a faster recovery. This two-way communication fosters a more interactive and engaging healthcare experience.

Moreover, the act of preparing questions in advance encourages users to actively participate in their healthcare journey. It empowers them to take ownership of their health by prompting discussions that may not have been considered during the initial consultation. As a result, this method not only improves patient engagement but also enhances treatment adherence, as users are more likely to follow through with medical advice when they have a clear understanding of their condition and the associated care plan. Ultimately, by addressing the user's concerns and providing tailored information, the method enhances recovery outcomes and contributes to a more patient-centered approach to healthcare.

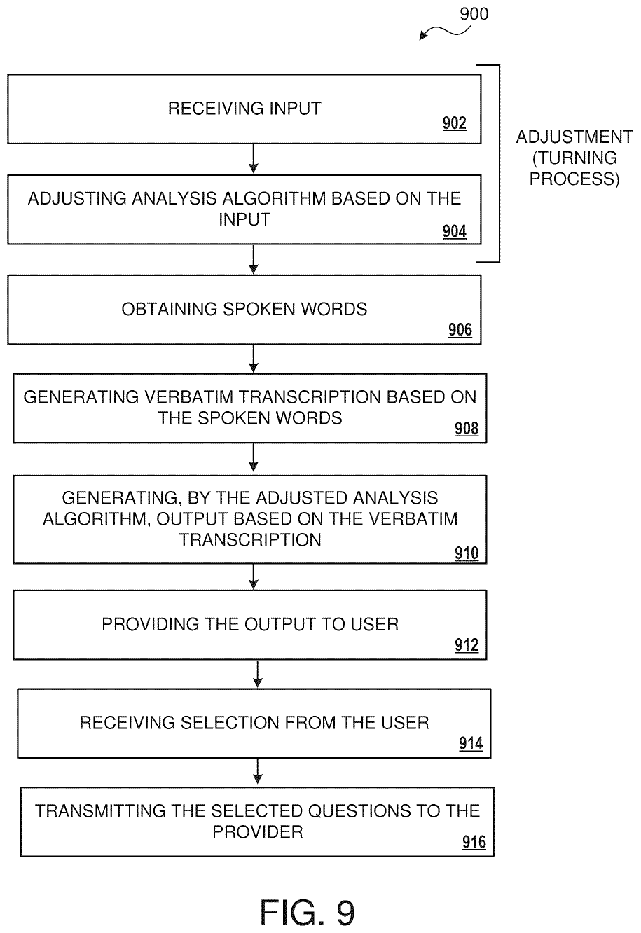

FIG. 9 is a flowchart of an example arrangement of operations for a method 900 of providing broadly understood output tailored to a specific reading level. The method 900 may be performed by processing logic that may include hardware (circuitry, dedicated logic, data processing hardware, etc.), software (such as is run on a general purpose computer system or a dedicated machine), or a combination of both, which processing logic may be included in any computer system or device. For simplicity of explanation, methods described herein are depicted and described as a series of acts. However, acts in accordance with this disclosure may occur in various orders and/or concurrently, and with other acts not presented and described herein. Further, not all illustrated acts may be used to implement the methods in accordance with the disclosed subject matter. In addition, those skilled in the art will understand and appreciate that the methods may alternatively be represented as a series of interrelated states via a state diagram or events. Additionally, the methods disclosed in this specification are capable of being stored on an article of manufacture, such as a non-transitory computer-readable medium, to facilitate transporting and transferring such methods to computing devices. The term article of manufacture, as used herein, is intended to encompass a computer program accessible from any computer-readable device or storage media. Although illustrated as discrete blocks, various blocks may be divided into additional blocks, combined into fewer blocks, or eliminated, depending on the desired implementation.

The method 900, at operation 902, includes receiving (e.g., obtaining), by a data processing hardware, input associated with a user's reading level. For example, the user (e.g., patient, caregiver) or healthcare provider may provide their reading level based on the Flesch-Kincaid Grade Level and/or Flesch Reading Ease Score. The healthcare provider or the user may also provide the user's native language, education level, and/or the number of years the user has lived in an English-speaking country.

The method 900, at operation 904, includes adjusting, by the data processing hardware, analysis algorithm based on the input. For example, the analysis algorithm is adjusted to ensure that the translation it generates is targeted to the specified reading level, considering factors such as the user's provided reading level, the number of years the user have lived in an English-speaking country, or his or her education level. In another example, the algorithm is adjusted to produce translations in the user's native language. The adjustment can also account for both the user's education level and native language in combination. The method 900, at operation 906, includes obtaining, by the data processing hardware, spoken words (e.g., spoken words from a healthcare provider). As discussed, these spoken words (e.g., specialized language) may include medical terminology that is difficult for a patient to understand.

The method 900, at operation 908, includes generating, by the data processing hardware, a verbatim transcription based on the spoken words. Natural language processing (NLP) may be implemented for generating the verbatim transcription from the spoken words.

The method 900, at operation 910, includes generating, by the data processing hardware, output (e.g., translation) using the adjusted analysis algorithm (e.g., trained analysis algorithm) based on the verbatim transcriptions. As discussed, the analysis algorithm may be operable to translate (e.g., paraphrase) the verbatim transcription into a broadly understood language (e.g., plain language) based on the user's reading level. When the spoken words are in a first language (e.g., English) and the user's native language (e.g., Chinese) is different from the first language, the analysis algorithm may translate the verbatim transcription into a plain language in the user's native language (based on the user's education level). For data sharing purpose, the analysis algorithm also may be operable to translate (e.g., paraphrase) the verbatim transcription into a broadly understood language in the first language (e.g., English). As a result, the application server, the partners, and other users can access this user's information easily (e.g., by performing key word search in the first language) when the user determines to share his or her data (e.g., uploading the user's data in the first language to the application server).

The method 900, at operation 912, includes providing, by the data processing hardware, the output to the user (e.g., patient user, caregiver user). As discussed, the output of the analysis algorithm may be displayed to the user either on as a text output (e.g., a digital display of text) and/or audibly as an audio output, such as via a voice synthesizer. The output may include a doctor visit summary. Additionally, the output may provide information on health insurance coverage, such as whether the doctor visit is covered by the user's health care insurance. information. Additionally, the output may include one or more suggested questions for the user to ask their healthcare provider, based on the user's condition as described in the output and/or the verbatim transcription.

The method 900 further includes generating, by the data processing hardware, one or more suggested questions (that were not addressed in the output and/or verbatim transcription) for the user to ask their healthcare provider. These suggested questions are then provided alongside the output, creating a comprehensive communication tool for the user. For example, this operation can be performed between operation 910 and operation 912.

For example, based on the output and/or verbatim transcription, the analysis algorithm may generate one or more suggested questions for the user to ask the healthcare provider that may provide additional valuable information for the user. These questions may include: “How will I know if my infection is getting worse?”, “Are there foods that I shouldn't eat with medicines you are prescribing?”, or “Are there any activities I should avoid for a faster recovery?”

Once these suggested questions are provided to the user, they have the opportunity to follow up with their healthcare provider for further clarification or guidance. This follow-up may take various forms, such as scheduling an in-person appointment, utilizing telehealth services, or communicating through secure messaging platforms that many healthcare systems now offer. As described in operations 914 and 916 below, the user may ask the questions to the healthcare provider while reviewing the output. Such flexibility in communication channels allows the user to choose the most convenient option based on their circumstances and preferences.

In this follow-up interaction, the user can present the suggested questions to their healthcare provider, ensuring they receive comprehensive answers tailored to their individual situation. For instance, the healthcare provider can explain the signs of worsening infection in detail, provide dietary recommendations that promote healing, and clarify which activities should be limited to facilitate a faster recovery. This two-way communication fosters a more interactive and engaging healthcare experience, enhancing the overall quality of care.

Moreover, the act of preparing questions in advance encourages users to actively participate in their healthcare journey. It empowers them to take ownership of their health by prompting discussions that may not have been considered during the initial consultation. As a result, this method not only improves patient engagement but also enhances treatment adherence, as users are more likely to follow through with medical advice when they have a clear understanding of their condition and the associated care plan. Ultimately, by addressing the user's concerns and providing tailored information, the method enhances recovery outcomes and contributes to a more holistic approach to healthcare.

The method 900, at operation 914, includes receiving, by the data processing hardware, one or more selections of suggested questions from the user. For example, the user may select one or more suggested questions using a suitable method, such as touch screen selection or a verbal request like, “Ask the first and third questions to my doctor.” This feature allows for ease of use and ensures that users can quickly communicate their inquiries without unnecessary complexity.

The method 900, at operation 916, include transmitting, by the data processing hardware, the selected one or more questions to the healthcare provider via a suitable communication channel, such as secure messaging platforms that many healthcare systems offer. This ensures that the questions are delivered efficiently and securely, facilitating timely responses from the healthcare provider and enhancing the user's ability to obtain the necessary information to manage their health effectively.

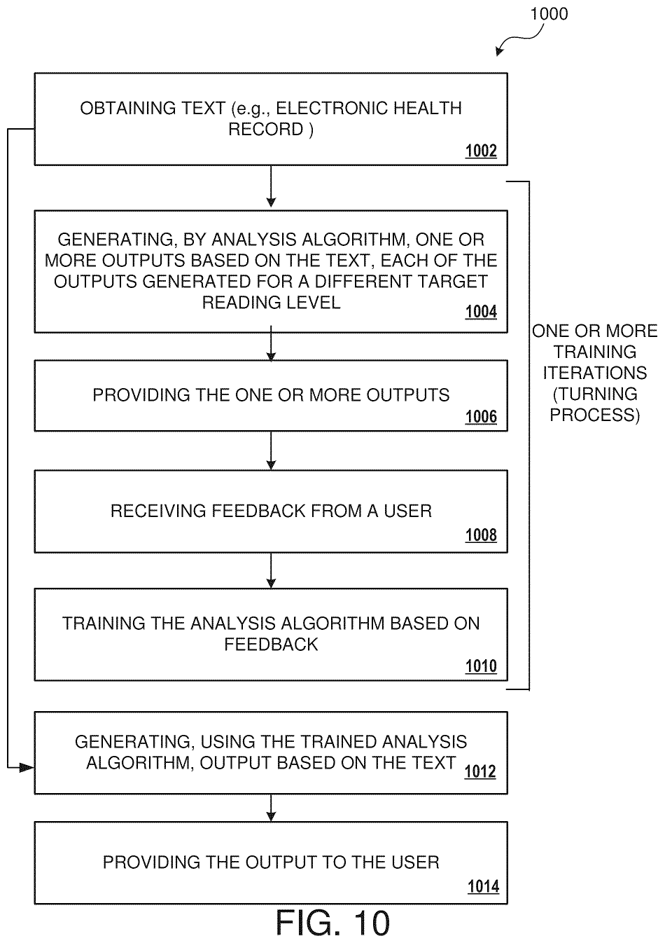

FIG. 10 is a flowchart of an example arrangement of operations for a method 1000 of providing broadly understood output tailored to a specific reading level. The method 1000 may be performed by processing logic that may include hardware (circuitry, dedicated logic, data processing hardware, etc.), software (such as is run on a general purpose computer system or a dedicated machine), or a combination of both, which processing logic may be included in any computer system or device. For simplicity of explanation, methods described herein are depicted and described as a series of acts. However, acts in accordance with this disclosure may occur in various orders and/or concurrently, and with other acts not presented and described herein. Further, not all illustrated acts may be used to implement the methods in accordance with the disclosed subject matter. In addition, those skilled in the art will understand and appreciate that the methods may alternatively be represented as a series of interrelated states via a state diagram or events. Additionally, the methods disclosed in this specification are capable of being stored on an article of manufacture, such as a non-transitory computer-readable medium, to facilitate transporting and transferring such methods to computing devices. The term article of manufacture, as used herein, is intended to encompass a computer program accessible from any computer-readable device or storage media. Although illustrated as discrete blocks, various blocks may be divided into additional blocks, combined into fewer blocks, or eliminated, depending on the desired implementation.

The method 1000, at operation 1002, includes obtaining (e.g., receiving), by a data processing hardware, text input (e.g., electronic health record such as patient record). As discussed, the text input (e.g., specialized language) may include medical terminology that is difficult for a patient to understand.

The method 1000, at operation 1004, during one or more training iterations, includes generating, by the data processing hardware, one or more outputs (e.g., translations) using analysis algorithm. As discussed, the analysis algorithm may be operable to translate (e.g., paraphrase) the text input to a broadly understood language. For example, the software application may translate the text input to a broadly understood language targeting a specific reading level (e.g., reading level based on Flesch-Kincaid Grade level, reading level based on Flesch Reading Ease Score, reading level based on education level). In another example, the software application may translate the text input into multiple translations, each translation tailored to a specific reading level (e.g., first translation aimed at a middle school graduate level and second translation aimed at a high school graduate level).

The method 1000, at operation 1006, during the one or more training iterations, includes providing, by the data processing hardware, the one or more outputs (e.g., translations) to a user (e.g., patient user, caregiver user). As discussed, the output of the analysis algorithm may be displayed to the user either on as a text output (e.g., a digital display of text) and/or audibly as an audio output, such as via a voice synthesizer.

The method 1000, at operation 1008, during the one or more training iterations, includes receiving, by the data processing hardware, feedback from the user. For example, the user may provide feedback indicating whether the output (e.g., translation) is too difficult or too easy to read. In another example, the user may indicate which of the multiple outputs (e.g., translations) is most suitable for reading.

The method 1000, at operation 1010, during the one or more training iterations, includes training (e.g., adjusting, turning), by the data processing hardware, the analysis algorithm based on the feedback received from the user. For example, when the user indicates that the output targeted at the high school graduate reading level is too difficult to read, the analysis algorithm is adjusted (e.g., trained) to target a lower reading level (e.g., a middle school graduate reading level). Similarly, when the user indicates that the output targeted at the high school graduate reading level is too easy to read, the analysis algorithm is adjusted (e.g., trained) to target a higher reading level (e.g., college graduate reading level). In another example, when the user indicates which of the multiple outputs is most suitable for reading, the analysis algorithm is adjusted (e.g., trained) to target the reading level corresponding to the preferred output (e.g., indicated translation).

The method 1000, at operation 1012, for subsequent translations, includes generating, by the data processing hardware, output (translation) using the trained analysis algorithm (e.g., adjusted analysis algorithm) based on subsequent verbatim texts.

The method 1000, at operation 1014, includes providing, by the data processing hardware, the output to the user (e.g., patient user, caregiver user). As discussed, the output of the analysis algorithm may be displayed to the user either on as a text output (e.g., a digital display of text) and/or audibly as an audio output, such as via a voice synthesizer. The output may include a doctor visit summary. Additionally, the output may provide information on health insurance coverage, such as whether the doctor visit is covered by the user's insurance. Additionally, the output may include one or more suggested questions for the user to ask their healthcare provider, based on the user's condition as described in the output and/or the verbatim transcription.

The method 1000 further includes generating, by the data processing hardware, one or more suggested questions (that were not addressed in the output and/or verbatim transcription) for the user to ask their healthcare provider. These suggested questions are then provided alongside the output, creating a comprehensive communication tool for the user. For example, this operation can be performed between operation 1012 and operation 1014.

For example, based on the output and/or verbatim transcription, the analysis algorithm may generate one or more suggested questions for the user to ask the healthcare provider that may provide additional valuable information for the user. These questions may include: “How will I know if my infection is getting worse?”, “Are there foods that I shouldn't eat with medicines you are prescribing?”, or “Are there any activities I should avoid for a faster recovery?”

Once these suggested questions are provided to the user, they have the opportunity to follow up with their healthcare provider for further clarification or guidance. This follow-up may take various forms, such as scheduling an in-person appointment, utilizing telehealth services, or communicating through secure messaging platforms that many healthcare systems offer. Such flexibility in communication channels allows the user to choose the most convenient option based on their circumstances and preferences.

In this follow-up interaction, the user can present the suggested questions to their healthcare provider, ensuring they receive comprehensive answers tailored to their individual situation. For instance, the healthcare provider can explain the signs of worsening infection in detail, provide dietary recommendations that promote healing, and clarify which activities should be limited to facilitate a faster recovery. This two-way communication fosters a more interactive and engaging healthcare experience.

Moreover, the act of preparing questions in advance encourages users to actively participate in their healthcare journey. It empowers them to take ownership of their health by prompting discussions that may not have been considered during the initial consultation. As a result, this method not only improves patient engagement but also enhances treatment adherence, as users are more likely to follow through with medical advice when they have a clear understanding of their condition and the associated care plan. Ultimately, by addressing the user's concerns and providing tailored information, the method enhances recovery outcomes and contributes to a more patient-centered approach to healthcare.

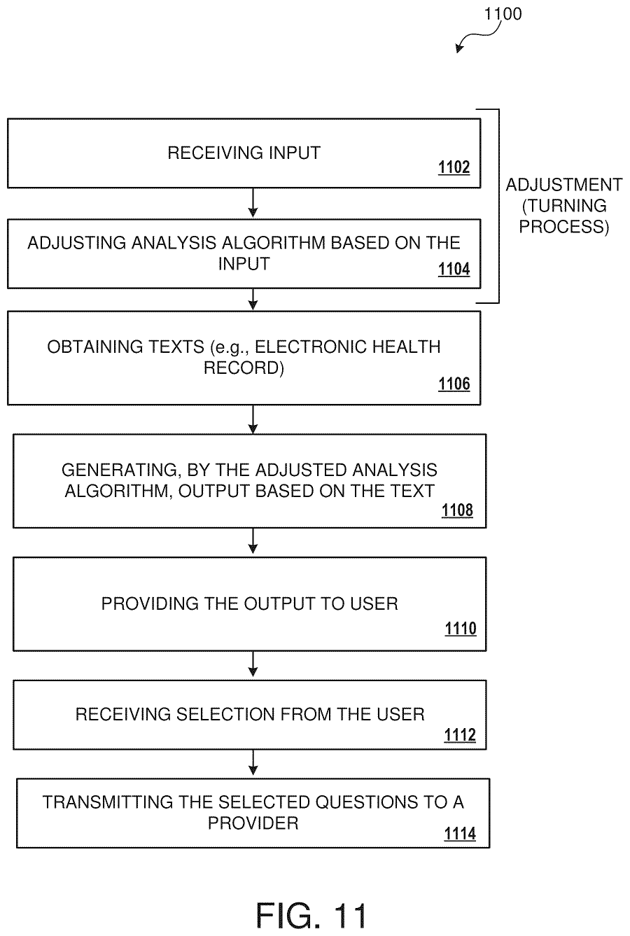

FIG. 11 is a flowchart of an example arrangement of operations for a method 1100 of providing broadly understood output tailored to a specific reading level. The method 1100 may be performed by processing logic that may include hardware (circuitry, dedicated logic, data processing hardware, etc.), software (such as is run on a general purpose computer system or a dedicated machine), or a combination of both, which processing logic may be included in any computer system or device. For simplicity of explanation, methods described herein are depicted and described as a series of acts. However, acts in accordance with this disclosure may occur in various orders and/or concurrently, and with other acts not presented and described herein. Further, not all illustrated acts may be used to implement the methods in accordance with the disclosed subject matter. In addition, those skilled in the art will understand and appreciate that the methods may alternatively be represented as a series of interrelated states via a state diagram or events. Additionally, the methods disclosed in this specification are capable of being stored on an article of manufacture, such as a non-transitory computer-readable medium, to facilitate transporting and transferring such methods to computing devices. The term article of manufacture, as used herein, is intended to encompass a computer program accessible from any computer-readable device or storage media. Although illustrated as discrete blocks, various blocks may be divided into additional blocks, combined into fewer blocks, or eliminated, depending on the desired implementation.

The method 1100, at operation 1102, includes receiving (e.g., obtaining), by a data processing hardware, input associated with a user's reading level. For example, the user (e.g., patient, caregiver) or healthcare provider may provide their reading level based on the Flesch-Kincaid Grade Level and/or Flesch Reading Ease Score. The healthcare provider or the user may also provide the user's native language, education level, and/or the number of years the user has lived in an English-speaking country.

The method 1100, at operation 1104, includes adjusting, by the data processing hardware, analysis algorithm based on the input. The analysis algorithm is adjusted to ensure that the translation it generates is targeted to the specified reading level, considering factors such as the user's provided reading level, the number of years the user have lived in an English-speaking country, or his or her education level. In another example, the algorithm is adjusted to produce translations in the user's native language. The adjustment can also account for both the user's education level and native language in combination.

The method 1100, at operation 1106, includes obtaining, by the data processing hardware, text input (e.g., electronic health record such as patient record, publication such as medical paper). As discussed, the text input (e.g., specialized language) may include medical terminology that is difficult for a patient to understand.

The method 1100, at operation 1108, includes generating, by the data processing hardware, output (e.g., translation) using the adjusted analysis algorithm (e.g., trained analysis algorithm) based on the text. As discussed, the analysis algorithm may be operable to translate (e.g., paraphrase) the text into a broadly understood language (e.g., plain language) based on the user's reading level. When the text is in a first language (e.g., English) and the user's native language (e.g., Chinese) is different from the first language, the analysis algorithm may translate the text into a plain language in the user's native language (based on the user's education level). For data sharing purpose, the analysis algorithm also may be operable to translate (e.g., paraphrase) the verbatim transcription into a broadly understood language in the first language (e.g., English). As a result, the application server, the partners, and other users can access this user's information easily (e.g., key word search in the first language) when the user determines to share his or her data (e.g., uploading the user's data in the first language to the application server).

The method 1100, at operation 1110, includes providing, by the data processing hardware, the output to the user (e.g., patient user, caregiver user). As discussed, the output of the analysis algorithm may be displayed to the user either on as a text output (e.g., a digital display of text) and/or audibly as an audio output, such as via a voice synthesizer. The output may include a plain language summary of a doctor visit summary (e.g., after-visit summary) tailored to the adjusted target reading level. Additionally, the output may include one or more suggested questions for the user to ask their healthcare provider, based on the user's condition as described in the output and/or the verbatim transcription.

The method 1100 further includes generating, by the data processing hardware, one or more suggested questions for the user to ask their healthcare provider. These suggested questions are then provided alongside the output, creating a comprehensive communication tool for the user. For example, this operation can be performed between operation 1108 and operation 1110.

For example, based on the output and/or verbatim transcription, the analysis algorithm may generate one or more suggested questions for the user to ask the healthcare provider that may provide additional valuable information for the user. These questions may include: “How will I know if my infection is getting worse?”, “Are there foods that I shouldn't eat with medicines you are prescribing?”, or “Are there any activities I should avoid for a faster recovery?”

Once these suggested questions are provided to the user, they have the opportunity to follow up with their healthcare provider for further clarification or guidance. This follow-up may take various forms, such as scheduling an in-person appointment, utilizing telehealth services, or communicating through secure messaging platforms that many healthcare systems now offer. As described in operations 1112 and 1114 below, the user may ask the questions to the healthcare provider while reviewing the output. Such flexibility in communication channels allows the user to choose the most convenient option based on their circumstances and preferences.

In this follow-up interaction, the user can present the suggested questions to their healthcare provider, ensuring they receive comprehensive answers tailored to their individual situation. For instance, the healthcare provider can explain the signs of worsening infection in detail, provide dietary recommendations that promote healing, and clarify which activities should be limited to facilitate a faster recovery. This two-way communication fosters a more interactive and engaging healthcare experience, enhancing the overall quality of care.

Moreover, the act of preparing questions in advance encourages users to actively participate in their healthcare journey. It empowers them to take ownership of their health by prompting discussions that may not have been considered during the initial consultation. As a result, this method not only improves patient engagement but also enhances treatment adherence, as users are more likely to follow through with medical advice when they have a clear understanding of their condition and the associated care plan. Ultimately, by addressing the user's concerns and providing tailored information, the method enhances recovery outcomes and contributes to a more patient-centered approach to healthcare.

The method 1100, at operation 1112, includes receiving, by the data processing hardware, one or more selections of suggested questions from the user. For example, the user may select one or more suggested questions using a suitable method, such as touch screen selection or a verbal request like, “Ask the first and third questions to my doctor.” This feature allows for ease of use and ensures that users can quickly communicate their inquiries without unnecessary complexity.

The method 1100, at operation 1114, include transmitting, by the data processing hardware, the selected one or more questions to the healthcare provider via a suitable communication channel, such as secure messaging platforms that many healthcare systems offer. This ensures that the questions are delivered efficiently and securely, facilitating timely responses from the healthcare provider and enhancing the user's ability to obtain the necessary information to manage their health effectively.

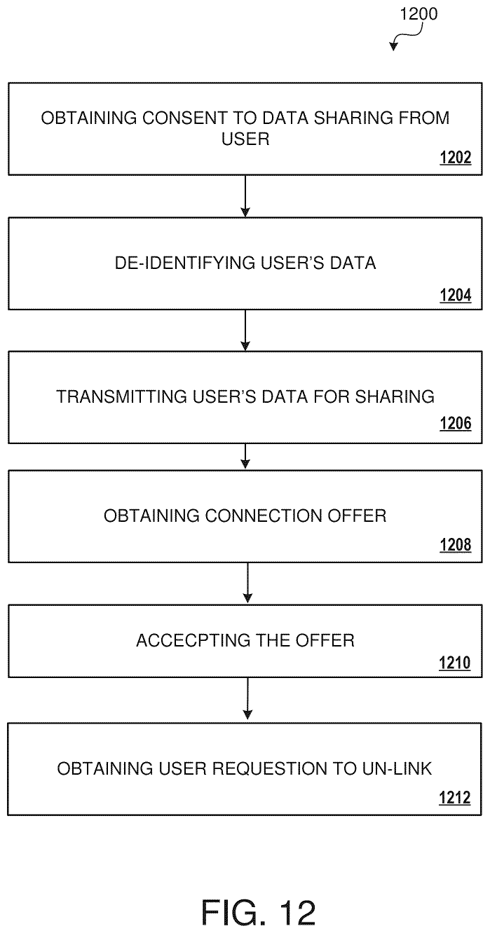

FIG. 12 is a flowchart of an example arrangement of operations for a method 1200 of establishing patient networks. The method 1200 may be performed by processing logic that may include hardware (circuitry, dedicated logic, data processing hardware, etc.), software (such as is run on a general purpose computer system or a dedicated machine), or a combination of both, which processing logic may be included in any computer system or device. For simplicity of explanation, methods described herein are depicted and described as a series of acts. However, acts in accordance with this disclosure may occur in various orders and/or concurrently, and with other acts not presented and described herein. Further, not all illustrated acts may be used to implement the methods in accordance with the disclosed subject matter. In addition, those skilled in the art will understand and appreciate that the methods may alternatively be represented as a series of interrelated states via a state diagram or events. Additionally, the methods disclosed in this specification are capable of being stored on an article of manufacture, such as a non-transitory computer-readable medium, to facilitate transporting and transferring such methods to computing devices. The term article of manufacture, as used herein, is intended to encompass a computer program accessible from any computer-readable device or storage media. Although illustrated as discrete blocks, various blocks may be divided into additional blocks, combined into fewer blocks, or eliminated, depending on the desired implementation.

The method 1200, at operation 1202, includes obtaining (e.g., receiving), by a data processing hardware, consent to data sharing from a user. As discussed, the consent to data sharing may be an explicit sharing permission from the user. The consent to data sharing may be an implicit sharing permission from the user.

The method 1200, at operation 1204, includes de-identifying, by the data processing hardware, the data (e.g., translation). The de-identified data are records that have a re-identification code and enough personally identifiable information removed or obscured so that the remaining information does not identify an individual and there is no basis to believe that the available information can be used to specifically identify any one individual.

The method 1200, at operation 1206, includes transmitting, by the data processing hardware, the de-identified data for sharing. As discussed, in some embodiments, the software application (e.g., software application on the user's smartphone) transmits (e.g., upload) the de-identified data to the software application server. The application server may store all the shared data in a database (e.g., database server).

The method 1200, at operation 1208, includes obtaining, by the data processing hardware, one or more connection offers (e.g., connection suggestions) from the software application server.

As discussed, the software application server determines commonalities in the key words in the shared data and offers users sharing the same commonalities an option to establish a 1:1 connection. In instances in which a first user and a second user have commonalities in the key words, the application server may offer the first user and the second user an option to establish a 1:1 connection. Alternatively, or additionally, the application server may offer the first user to be added to any user connections that the second user has already established, and for the second user to be added to any user connections that the first user has already established. As such, the application server may facilitate the formation of patient networks that may be linked by broadly understood medical conditions, treatments, or interests.

The method 1200, at operation 1210, includes accepting, by the data processing hardware, the offer from the software application server.

The method 1200, at operation 1212, includes obtaining a user request to un-link connections by the data processing hardware. Upon receiving a user request, the software application (e.g., software application on the user's smartphone) unlinks the specified connection at any time as desired by the user.

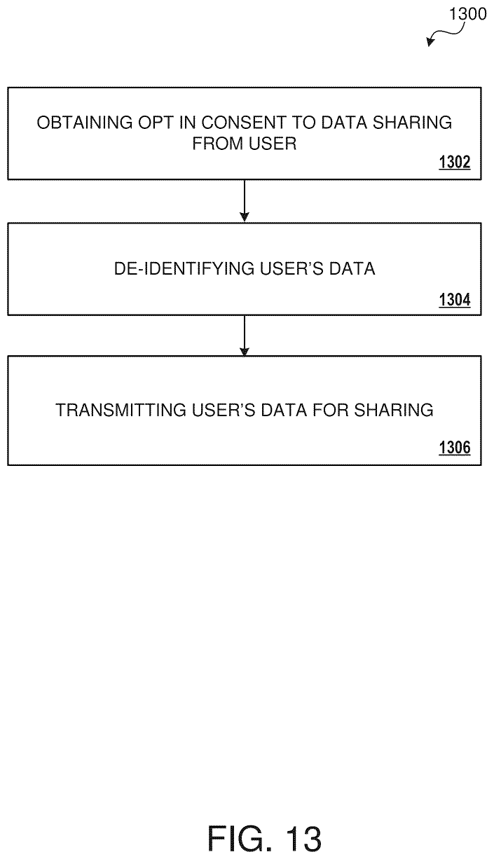

FIG. 13 is a flowchart of an example arrangement of operations for a method 1300 of sharing user's data (e.g., translated health record) with one or more partners. The method 1300 may be performed by processing logic that may include hardware (circuitry, dedicated logic, data processing hardware, etc.), software (such as is run on a general purpose computer system or a dedicated machine), or a combination of both, which processing logic may be included in any computer system or device. For simplicity of explanation, methods described herein are depicted and described as a series of acts. However, acts in accordance with this disclosure may occur in various orders and/or concurrently, and with other acts not presented and described herein. Further, not all illustrated acts may be used to implement the methods in accordance with the disclosed subject matter. In addition, those skilled in the art will understand and appreciate that the methods may alternatively be represented as a series of interrelated states via a state diagram or events. Additionally, the methods disclosed in this specification are capable of being stored on an article of manufacture, such as a non-transitory computer-readable medium, to facilitate transporting and transferring such methods to computing devices. The term article of manufacture, as used herein, is intended to encompass a computer program accessible from any computer-readable device or storage media. Although illustrated as discrete blocks, various blocks may be divided into additional blocks, combined into fewer blocks, or eliminated, depending on the desired implementation.

The method 1300, at operation 1302, includes obtaining (e.g., receiving), by a data processing hardware, consent to data sharing from a user. As discussed, the consent to data sharing may be an explicit sharing permission from the user. The consent to data sharing may be an implicit sharing permission from the user.

The method 1300, at operation 1304, includes de-identifying, by the data processing hardware, the data (e.g., translation). The de-identified data are records that have a re-identification code and enough personally identifiable information removed or obscured so that the remaining information does not identify an individual and there is no basis to believe that the available information can be used to specifically identify any one individual.

The method 1300, at operation 1306, includes transmitting, by the data processing hardware, the de-identified data for sharing. As discussed, in some embodiments, the software application (e.g., software application on the user's smartphone) transmits (e.g., upload) the de-identified data to the software application server. The application server may store all the shared data in a database (e.g., database server). The stored data can be accessed by one or more partners. The partners may include one or more of the following: biopharma companies, healthcare providers, payers, clinical trial sponsors, government health agencies, academic institutions, patient advocacy organizations, or public health agencies. The stored data can be accessed by other users.

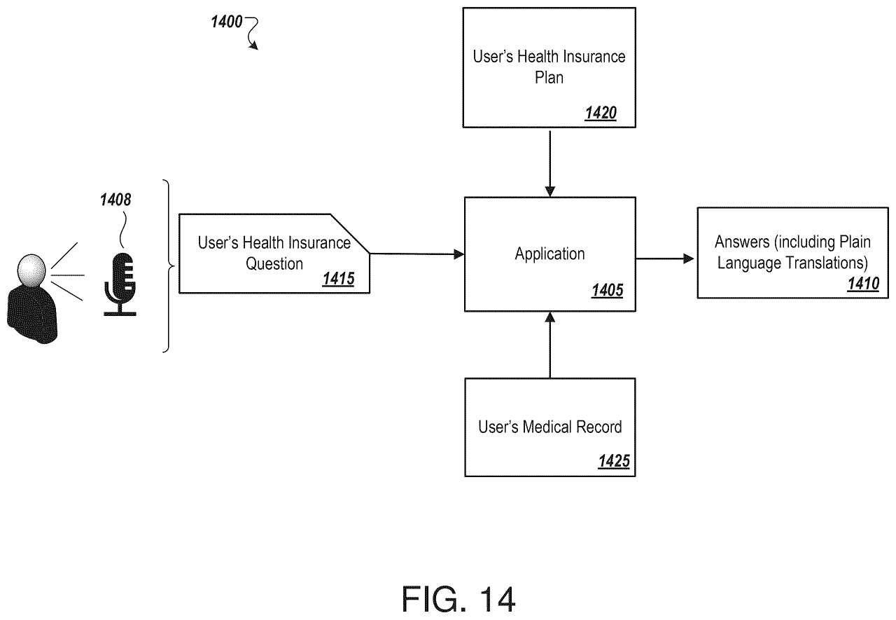

FIG. 14 is a diagram of system 1400, which is operable to answer the user's questions related to their health insurance plan by analyzing the user's health insurance plan (e.g., health insurance policy document associated with the user's health insurance plan) in accordance with some implementations of the disclosure. In other words, system 1400 is designed to receive health insurance-related questions (e.g., health insurance coverage questions) and provide answers based on an analysis of the user's health insurance plan. To enhance the accuracy of the responses, system 1400 may also incorporate an analysis of both the user's health insurance plan and their medical record.

System 1400 may include a software application 1405 configured to obtain the user's questions related to their insurance plan 1415 and to provide answers 1410 based on an analysis of the user's health insurance plan 1420 using an algorithm (e.g., a combination of algorithms). In some examples, the user's medical record 1425 may also be used when analyzing the user's health insurance plan 1420.

Software application 1405 may be implemented on smartphones, wearables, desktop computers, laptop computers, and/or other computing hardware platforms. System 1400 may also be operable to perform one or more operations, including obtaining (e.g., receiving) the user's questions (e.g., via verbal, keyboard, or touchpad input), analyzing the user's health insurance plan 1420 (optionally along with the user's medical record 1425), and providing answers 1410 (e.g., answer in plain language, answer including plain language) to the user. These answers 1410 may be presented in broadly understood language (e.g., plain language), tailored to the user's target reading level by the trained algorithm described above in FIG. 1-FIG. 13. The answers 1410 may be displayed as text (e.g., on a digital screen) and/or delivered audibly via a voice synthesizer.

System 1400 may use a recording device 1408 to obtain the user's health insurance questions. Recording device 1408 may be a peripheral recording device that is separate from system 1400. Additionally, the recording 1408 device may be connected to the system 1400, incorporated into the system 1400 and/or integral with the system 1400. Software application 1405 may use the recording device 1408. The software application 1405 may use an algorithm (e.g., combination of algorithms) to translate the user's health insurance questions into language commonly used by the health insurance industry (e.g., terminology commonly used in health insurance policy). The software application 1405 may perform analyzing the user's health insurance plan 1420 (e.g., health insurance policy document associated with the user's health insurance plan) based on the translated questions to determine the answers 1410. The software application 1405 may translate the answers 1410 into the broadly understood language from the language commonly used by the health insurance industry.

For example, the user may ask, “Is the breast cancer check covered by my health insurance?” The application 1405 receives the question and translates it into “Is the screening mammogram covered by my health insurance?” using the algorithm. The application 1405 further analyzes the user's insurance plan to determine whether the screening mammogram is covered and provides answer 1410 to the user. For instance, the application 1405 may respond, “One screening mammogram may be covered for every calendar year.” Alternatively, it may state, “One breast check may be covered for every calendar year,” using plain language tailored to the user's reading level.

In another example, the user may ask, “Is a weight loss drug covered by my health insurance?” The application 1405 receives the question and translates it into “Are prescription medications for obesity treatment covered by my health insurance?” using the algorithm. The application 1405 then analyzes the user's health insurance plan 1420 to determine whether the prescription medications for obesity treatment are covered. It may provide an answer such as, “Prescription medications for obesity treatment are covered if you have a diagnosis code E66.3 from your doctor and obtain a pre-approval from the health insurance provider.” Another possible response could be, “If your doctor prescribes a weight-loss drug and provides a diagnosis code of E66.3 (indicating that you are overweight) and received a pre-approval from the health insurance provider, your health insurance may cover it” using plain language tailored to the user's reading level.

In another example, the user may ask, “If I get the breast cancer check this month, is it covered by my health insurance?” The application 1405 receives the question and translates it into, “Is the screening mammogram covered by my health insurance this month?” using the algorithm. The application 1405 further analyzes the user's medical record and insurance policy to determine whether the screening mammogram is covered this month. For instance, if the application 1405 determines that one screening mammogram is covered per calendar year and finds no record of a screening mammogram in the user's medical record for this year, it may respond, “The screening mammogram may be covered for this month.” Alternatively, it may state, “The breast check may be covered for this month,” using plain language tailored to the user's reading level.

In another example, the software application 1405 may use the algorithm to translate the user's health insurance questions into language commonly used by the health insurance industry (e.g., terminology commonly used in health insurance policy). The software application 1405 may identify one or more relevant sections in the user's health insurance plan (e.g., health insurance policy document associated with the user's health insurance plan) based on the translated questions and translate the relevant sections into the broadly understood language. In this case, the answers 1410 includes the relevant section of the user's health insurance policy document in the broadly understood language.

As described above, the application 1405 can provide answers to users' health insurance questions, specifying conditions needed for approval from the health insurance provider, such as prescriptions, pre-approval from the health insurance provider, or diagnosis codes. As described above, the answers may include the relevant section of the user's health insurance policy document in the broadly understood language.

This functionality helps users understand what is required to access their insurance benefits effectively.

The system 1400 is configured to answer users' health insurance questions by analyzing their insurance policies or their insurance policies alongside with medical records. Utilizing a software application, it translates user inquiries into industry-standard terminology and provides tailored responses. For example, it can convert a question about breast cancer checks into a query about screening mammograms, ensuring clarity. The system also specifies conditions for insurance coverage, helping users understand what is required for approval from their health insurance provider.

Additionally, the system's ability to tailor responses to the user's reading level enhances comprehension, making it easier for users to navigate their health insurance options. The system 1400 is designed to simplify complex insurance terminology, fostering clear communication that empowers users to make well-informed decisions regarding their healthcare coverage. By translating intricate language into understandable terms, the system enhances users' ability to navigate their insurance options confidently.

For your system 1400, one or more algorithms can be utilized to analyze health insurance questions and translate them into industry-standard terminology. For example, the one or more algorithms may include Natural Language Processing (NLP) Algorithms techniques such as tokenization, named entity recognition (NER), and part-of-speech tagging to parse user questions and identify key terms relevant to health insurance. The one or more algorithms may include semantic similarity algorithms that measure the semantic similarity between user queries and standard insurance terminology, such as word embeddings (e.g., Word2Vec, GloVe) or transformer models (e.g., BERT). The one or more algorithms may include rule-based algorithms. The rule-based algorithms can create a set of rules that map common user questions to standardized terms in health insurance. This approach can be combined with NLP for better accuracy. The one or more algorithms may include machine learning algorithms such as decision trees or random forests. The decision trees or random forests can analyze the user's insurance policy and medical records to determine coverage based on various input features, such as diagnosis codes or serve types. Reinforcement learning can refine response accuracy over time based on user feedback, optimizing the system's ability to answer questions. Combining these approaches creates a comprehensive system that delivers precise and actionable responses to health insurance inquiries, which may include plain language translations of insurance documents.

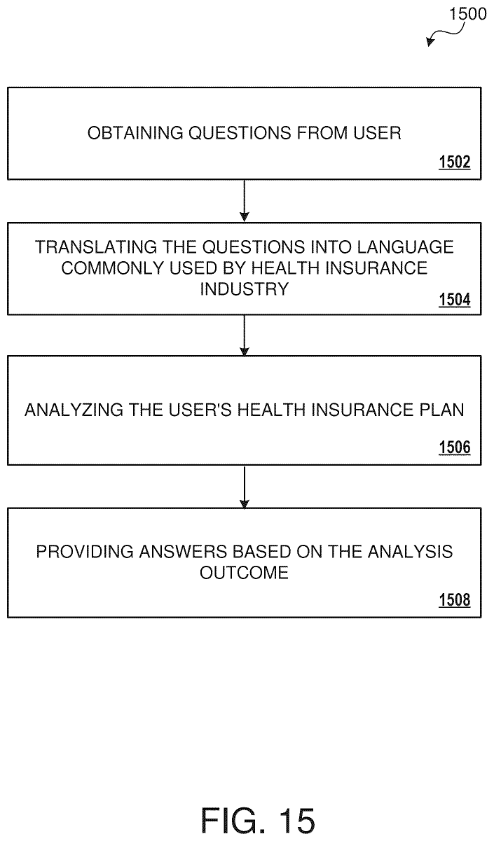

FIG. 15 is a flowchart of an example arrangement of operations for a method 1500 of providing answers to a user's insurance related questions in accordance with some implementations. The method 1500 may be performed by processing logic that may include hardware (circuitry, dedicated logic, data processing hardware, etc.), software (such as is run on a general purpose computer system or a dedicated machine), or a combination of both, which processing logic may be included in any computer system or device. For simplicity of explanation, methods described herein are depicted and described as a series of acts. However, acts in accordance with this disclosure may occur in various orders and/or concurrently, and with other acts not presented and described herein. Further, not all illustrated acts may be used to implement the methods in accordance with the disclosed subject matter. In addition, those skilled in the art will understand and appreciate that the methods may alternatively be represented as a series of interrelated states via a state diagram or events. Additionally, the methods disclosed in this specification are capable of being stored on an article of manufacture, such as a non-transitory computer-readable medium, to facilitate transporting and transferring such methods to computing devices. The term article of manufacture, as used herein, is intended to encompass a computer program accessible from any computer-readable device or storage media. Although illustrated as discrete blocks, various blocks may be divided into additional blocks, combined into fewer blocks, or eliminated, depending on the desired implementation.

The method 1500, at operation 1502, includes obtaining (e.g., receiving), by a data processing hardware, questions from a user. System may use a recording device to obtain the user's health insurance question. For example, the user's health insurance question is “Is the breast cancer check covered by my health insurance?”

The method 1500, at operation 1504, includes translating, by the data processing hardware, the question from the user into language commonly used by the health insurance industry (e.g., terminology commonly used in health insurance policy). For example, the question is translated to “Is the screening mammogram covered by my health insurance?” using the algorithm.

The method 1500, at operation 1506, includes analyzing, by the data processing hardware, the user's health insurance plan (e.g., health insurance policy document associated with the user's health insurance plan) based on translated question. For example, the analyzing the health insurance plan includes identifying, by the data processing hardware, one or more relevant sections in the health insurance plan based on the translated question, and translating, by the data processing hardware, the identified relevant sections from the language commonly used by the health insurance industry into broadly understood language. In another example, the analyzing the health insurance plan includes determining, by the data processing hardware, an answer within the health insurance plan (e.g., health insurance policy document associated with the user's health insurance plan) based on the translated question and translating, by the data processing hardware, the answer from the language commonly used by the health insurance industry into the broadly understood language.

The method 1500, at operation 1508, includes providing, by the data processing hardware, answers (e.g., relevant sections in the broadly understood language, answer in the broadly understood language) based on the outcome from the analysis using the algorithm.

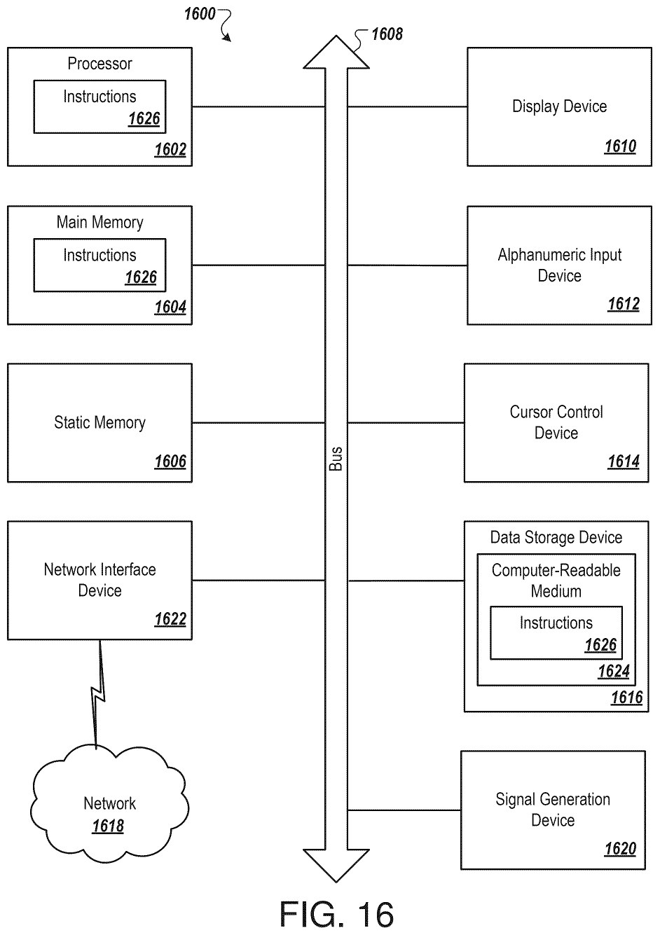

FIG. 16 illustrates a diagrammatic representation of a machine in the example form of a computing device 1600 within which a set of instructions, for causing the machine to perform any one or more of the methods discussed herein, may be executed. The computing device 1600 may include a mobile phone, a smart phone, a netbook computer, a rackmount server, a router computer, a server computer, a personal computer, a mainframe computer, a laptop computer, a tablet computer, a desktop computer etc., within which a set of instructions, for causing the machine to perform any one or more of the methods discussed herein, may be executed. In alternative embodiments, the machine may be connected (e.g., networked) to other machines in a local area network (LAN), an intranet, an extranet, or the Internet. The machine may operate in the capacity of a server machine in client-server network environment. The machine may include a personal computer (PC), a set-top box (STB), a server, a network router, switch or bridge, or any machine capable of executing a set of instructions (sequential or otherwise) that specify actions to be taken by that machine. Further, while only a single machine is illustrated, the term “machine” may also include any collection of machines that individually or jointly execute a set (or multiple sets) of instructions to perform any one or more of the methods discussed herein.

The example computing device 1600 includes a processor (e.g., a data processing hardware) 1602, a main memory 1604 (e.g., read-only memory (ROM), flash memory, dynamic random access memory (DRAM) such as synchronous DRAM (SDRAM)), a static memory 1606 (e.g., flash memory, static random access memory (SRAM)) and a data storage device 1616, which communicate with each other via a bus 1608.

Processing device 1602 represents one or more general-purpose processing devices such as a microprocessor, central processing unit, or the like. More particularly, the processing device 1602 may include a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 1602 may also include one or more special-purpose processing devices such as an application specific integrated circuit (ASIC), a field programmable gate array (FPGA), a digital signal processor (DSP), network processor, or the like. The processing device 1602 is configured to execute instructions 1626 for performing the operations and steps discussed herein.

The computing device 1600 may further include a network interface device 1622 which may communicate with a network 1618. The computing device 1400 also may include a display device 1610 (e.g., a liquid crystal display (LCD) or a cathode ray tube (CRT)), an alphanumeric input device 1612 (e.g., a keyboard), a cursor control device 1614 (e.g., a mouse) and a signal generation device 1620 (e.g., a speaker). In at least one embodiment, the display device 1610, the alphanumeric input device 1612, and the cursor control device 1614 may be combined into a single component or device (e.g., an LCD touch screen).

The data storage device 1616 may include a computer-readable storage medium 1624 on which is stored one or more sets of instructions 1626 embodying any one or more of the methods or functions described herein. The instructions 1626 may also reside, completely or at least partially, within the main memory 1604 and/or within the processing device 1602 during execution thereof by the computing device 1600, the main memory 1604 and the processing device 1602 also constituting computer-readable media. The instructions may further be transmitted or received over a network 1618 via the network interface device 1622.

While the computer-readable storage medium 1624 is shown in an example embodiment to be a single medium, the term “computer-readable storage medium” may include a single medium or multiple media (e.g., a centralized or distributed database and/or associated caches and servers) that store the one or more sets of instructions. The term “computer-readable storage medium” may also include any medium that is capable of storing, encoding or carrying a set of instructions for execution by the machine and that cause the machine to perform any one or more of the methods of the present disclosure. The term “computer-readable storage medium” may accordingly be taken to include, but not be limited to, solid-state memories, optical media and magnetic media.

While preferred embodiments of the present invention have been shown and described herein, it will be obvious to those skilled in the art that such embodiments are provided by way of example only. Numerous variations, changes, and substitutions will now occur to those skilled in the art without departing from the scope of the invention. For example, the use of comprise, or variants such as comprises or comprising, includes a stated integer or group of integers but not the exclusion of any other integer or group of integers. It should be understood that various alternatives to the embodiments of the invention described herein may be employed in practicing the invention. It is intended that any claims presented at any time in this application define the scope of the invention and that methods and structures within the scope of these claims and their equivalents are covered thereby.

What is claimed is:

1. A method comprising:
obtaining, at data processing hardware, a first input, the first input including medical information associated with a user, the medical information associated with the user including one or more medical terms;
generating, by the data processing hardware, a first output based on the first input using an algorithm, the first output including a first interpretation of the medical information associated with the user; and
providing, by the data processing hardware, the first output to the user;
wherein the first interpretation includes a first result of paraphrasing the one or more medical terms in the first input into broadly understood language.

2. The method of claim 1, wherein the medical information associated with the user is a transcription based on spoken words from a healthcare provider.

3. The method of claim 1, wherein the medical information of the user includes an electronic health record (EHR) of the user or a written communication from a healthcare provider.

4. The method of claim 1, wherein providing the first output to the user includes displaying the first interpretation to the user.

5. The method of claim 1, wherein providing the first output to the user includes:
converting the first interpretation into a first synthesized voice; and
playing the first synthesized voice.

6. The method of claim 1, wherein the algorithm includes one or more algorithms, the one or more algorithms including natural language processing (NLP).

7. The method of claim 1, further comprising:
obtaining, at the data processing hardware, a second input, the second input including medical information not associated with the user, the medical information not associated with user including one or more medical terms;
generating, by the data processing hardware, a second output based on the second input using the algorithm, the second output including a second interpretation of the medical information not associated with the user; and
providing, by the data processing hardware, the second output to the user,
wherein the second interpretation includes a second result of paraphrasing the one or more medical terms in the second input into broadly understood language.

8. The method of claim 7, wherein the second input includes a medical publication.

9. The method of claim 1, wherein the algorithm is trained by:
performing one or more training iterations, the one or more training iterations including:
obtaining, at the data processing hardware, a training input, the training input including training medical information;
generating, by the data processing hardware, a plurality of training outputs based on the training input using the algorithm, the plurality of training outputs including a second interpretation of the training medical information targeted at a first reading level and a third interpretation of the training medical information targeted at a second reading level;
providing, by the data processing hardware, the plurality of training outputs to the user;
receiving, by the data processing hardware, feedback from the user on the training outputs; and
training, by the data processing hardware, the algorithm based on the feedback received from the user.

10. The method of claim 9, wherein the training medical information is the medical information associated with the user.

11. The method of claim 9, wherein:
the first reading level is associated with a first Flesch Reading Ease Score and the second reading level is associated with a second Flesch Reading Ease Score.

12. The method of claim 9, wherein:
the first reading level is associated with a first Flesch-Kincaid Grade Level and the second reading level is associated with a second Flesch-Kincaid Grade Level.

13. The method of claim 9, wherein the receiving the feedback from the user on the training outputs includes receiving an indication of a preferred output by the user.

14. The method of claim 9, wherein the training the algorithm based on the feedback includes adjusting the algorithm to target a reading level corresponding to the preferred output.

15. The method of claim 1, further comprising:
generating, by the data processing hardware, one or more suggested questions that provide additional valuable information for the user; and
incorporating, by the data processing hardware, the one or more suggested questions into the first output.

16. A method, comprising:
obtaining, at data processing hardware, a consent to data sharing from a user;
de-identifying, by the data processing hardware, data of the user;
transmitting, by the data processing hardware, the de-identified data to a server; and
receiving, by the data processing hardware, a connection suggestion from the server,
wherein the data includes a first interpretation of medical information associated with the user,
wherein the medical information associated with the user includes one or more medical terms, and
wherein the first interpretation includes a first result of paraphrasing the one or more medical terms in the medical information associated with the user into broadly understood language.

17. The method of claim 16, wherein the consent to data sharing is an explicit sharing permission or an implicit sharing permission.

18. The method of claim 16, wherein the medical information associated with the user includes after-visit summary provided by a healthcare provider.

19. The method of claim 16, wherein the server stores the de-identified data and provides access to the de-identified data to one or more partners, the one or more partners including at least one of: biopharma companies, healthcare providers, payers, clinical trial sponsors, government health agencies, academic institutions, or patient advocacy organizations.

20. The method of claim 16, wherein:
the server determines one or more commonalities based on one or more keywords found in the data of the user and other users' data, and
the server transmits connection suggestion from the server based on the one or more commonalities found.

21. The method of claim 20, wherein the keywords are non-medical terms.

22. A method, comprising:
obtaining, at data processing hardware, first de-identified data from a first software application and second de-identified data from a second software application;
storing, by the data processing hardware, the first de-identified data and the second de-identified data;
determining, by the data processing hardware, one or more commonalities based on one or more keywords found in the first de-identified data and the second de-identified data; and
in response to a determination that the first de-identified data and the second de-identified data share one or more commonalities, transmitting, by the data processing hardware, a connection offer to the first software application and the second software application.

23. The method of claim 21, wherein the keywords are non-medical terms.

24. The method of claim 22, further comprising:
providing, by the data processing hardware, access to the first de-identified data and the second de-identified data to one or more partners, the one or more partners including at least one of following: biopharma companies, healthcare providers, payers, clinical trial sponsors, government health agencies, academic institutions, or patient advocacy organizations.

25. A method comprising:
obtaining, at data processing hardware, a first question from a user, the first question about user's health insurance plan;
translating, by data processing hardware, the first question into language commonly used by health insurance industry using an algorithm;
analyzing, by the data processing hardware, health insurance document associated with the user's health insurance plan based on the first translated question; and
providing, by the data processing hardware, a first answer based on the analysis outcome.

26. The method of claim 25, wherein:
the first question from the user is a health insurance coverage inquiry;
the analyzing the health insurance document includes:
identifying, by the data processing hardware, one or more relevant sections in the health insurance document based on the first translated question;
translating, by the data processing hardware, the identified relevant sections into broadly understood language; and
the first answer includes the translated relevant sections.

27. The method of claim 25, wherein:
the first question from the user is a health insurance coverage inquiry;
the analyzing the health insurance document includes:
determining, by the data processing hardware, an answer within the health insurance document based on the first translated question, the answer being in the language commonly used by the health insurance industry;
translating, by the data processing hardware, the answer into broadly understood language; and
the first answer is based on the translated version of the answer.
